(19) United States
(12) Patent Application Publication
(10) Pub. No.: US 2025/0186538 A1
(43) Pub. Date: Jun. 12, 2025

(54) BIVALIRUDIN COMPOUNDS

(71) Applicant: The General Hospital Corporation, Boston, MA (US)

(72) Inventors: Jonathan Carlson, Boston, MA (US); Hannes Mikula, Pixendorf (AT)

(21) Appl. No.: 18/844,839

(22) PCT Filed: Apr. 3, 2023
(86) PCT No.: PCT/US2023/017270

§ 371 Date: Sep. 6, 2024

Related U.S. Application Data
(60) Provisional application No. 63/328,737, filed on Apr. 7, 2022.

(51) Int. Cl. A61K 38/10; A61K 38/07; A61P 7/02; C07K 14/815

(52) U.S. Cl. CPC A61K 38/10; A61K 38/07; A61P 7/02; C07K 14/815

(57) ABSTRACT

The present application provides compounds and methods for reducing coagulation.

### CLAIM OF PRIORITY

This application claims priority to U.S. Provisional Patent Application Ser. No. 63/328,737, filed on Apr. 7, 2022, the entire contents of which are hereby incorporated by reference.

### TECHNICAL FIELD

This invention relates to bivalirudin and related compounds containing a trans-cyclooctene, tetrazine, and/or cycloocta[d]pyridazine moieties in their chemical structures. These compounds are capable of retaining their anticoagulant activity for a pre-determined period of time. After the time-limited period of activity, the intramolecular scission reaction leads decomposition of the compound producing molecular fragments lacking any anticoagulant activity.

### BACKGROUND

Excessive clotting, also known as thrombophilia or hypercoagulation, present significant socioeconomic burden. Blood clots can form in vital organs or travel to them, including the heart and brain. This can cause serious health problems, even death. A blood clot in the heart or lungs can cause a heart attack or a pulmonary embolism.

### SUMMARY

The present disclosure provides, inter alia, anticoagulant compounds and methods that advantageously allow to spatially restrict the scope of anticoagulation to affected regions within the circulation. The compounds achieve selective local or locoregional delivery downstream from the site of infusion. Including bioorthogonal, self-destructing moiety within the chemical structure of bivalirudin allows to modify half-life of the compound for spatiotemporal control to localize activity and/or mitigate systemic toxicity and other undesired events.

In one general aspect, the present disclosure provides a compound of Formula (I):

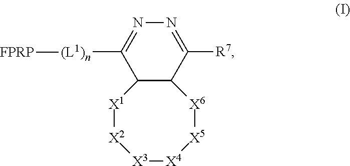

or a pharmaceutically acceptable salt thereof, wherein L¹, n, R⁷, X¹, X², X³, X⁴, X⁵, and X⁶ are as defined herein.

In another general aspect, the present disclosure provides a compound of Formula (II):

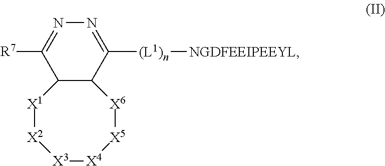

or a pharmaceutically acceptable salt thereof, wherein R⁷, L¹, n, X¹, X², X³, X⁴, X⁵, and X⁶ are as defined herein.

In yet another general aspect, the present disclosure provides a pharmaceutical composition comprising a compound of Formula (I) or Formula (II), or a pharmaceutically acceptable salt thereof, and a pharmaceutically acceptable carrier.

In yet another general aspect, the present disclosure provides a method of treating or preventing coagulation and/or blood clotting in a subject in need thereof, the method comprising administering to the subject a therapeutically effective amount of a compound of Formula (I) or Formula (II), or a pharmaceutically acceptable salt thereof, or a pharmaceutical composition comprising same.

In yet another general aspect, the present disclosure provides a compound having Formula (III):

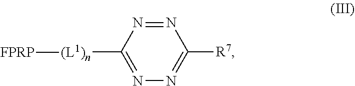

or a pharmaceutically acceptable salt thereof, wherein L¹, n, and R⁷ are as described herein.

In yet another general aspect, the present disclosure provides a compound of Formula (IV):

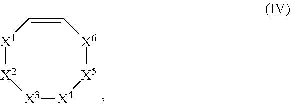

or a pharmaceutically acceptable salt thereof, wherein X¹, X², X³, X⁴, X⁵, and X⁶ are as defined herein.

In yet another general aspect, the present disclosure provides a pharmaceutical composition comprising a compound of Formula (III) or Formula (IV), or a pharmaceutically acceptable salt thereof, and a pharmaceutically acceptable carrier.

In yet another general aspect, the present disclosure provides a method of making a compound of Formula (I), or a pharmaceutically acceptable salt thereof, as described herein, the method comprising reacting a compound of Formula (III), or a pharmaceutically acceptable salt thereof, as described herein, with a compound of formula (IV), or a pharmaceutically acceptable salt thereof, as described herein, to obtain the compound of Formula (I), or a pharmaceutically acceptable salt thereof.

In yet another general aspect, the present disclosure provides a method of treating or preventing coagulation and/or blood clotting in a subject in need thereof, the method comprising administering to the subject a therapeutically effective amount of a compound of Formula (III), or a pharmaceutically acceptable salt thereof, as described herein, or a pharmaceutical composition comprising same, in combination with a therapeutically effective amount of a compound of Formula (IV), or a pharmaceutically acceptable salt thereof, as described herein, or a pharmaceutical composition comprising same.

In yet another general aspect, the present disclosure provides a compound of Formula (V):

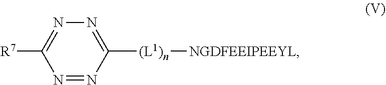

or a pharmaceutically acceptable salt thereof, wherein R⁷, L¹, and n are as described herein.

In yet another general aspect, the present disclosure provides a compound of Formula (VI):

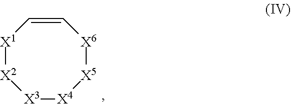

or a pharmaceutically acceptable salt thereof, wherein X¹, X², X³, X⁴, X⁵, and X⁶ are as defined herein.

In yet another general aspect, the present disclosure provides a pharmaceutical composition comprising a compound of Formula (V) or Formula (VI), or a pharmaceutically acceptable salt thereof, and a pharmaceutically acceptable carrier.

In yet another general aspect, the present disclosure provides a method of making a compound of Formula (II), or a pharmaceutically acceptable salt thereof, as described herein, the method comprising reacting a compound of Formula (V), or a pharmaceutically acceptable salt thereof, as described herein, with a compound of formula (VI), or a pharmaceutically acceptable salt thereof, as described herein, to obtain the compound of Formula (II), or a pharmaceutically acceptable salt thereof.

In yet another general aspect, the present disclosure provides a method of treating or preventing coagulation and/or blood clotting in a subject in need thereof, the method comprising administering to the subject a therapeutically effective amount of a compound of Formula (V), or a pharmaceutically acceptable salt thereof, as described herein, or a pharmaceutical composition of comprising same, in combination with a therapeutically effective amount of a compound of Formula (VI), or a pharmaceutically acceptable salt thereof, as described herein, or a pharmaceutical composition comprising same.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 shows administration strategies for spatiotemporal anticoagulation control. By combining control parameters (e.g., concentration, pH, time, chemical functionality) to regulate tetrazine/trans-cyclooctene “Tz/TCO” click and cleavage kinetics, multiple mechanisms for spatially restricted anticoagulation (AC) can be designed. Central infusion: intact TCO/Tz linked fragments of bivalirudin (click-Bival) are prepared at selected conditions—concentrations, mixing and delivery are tuned such that complete reaction is achieved (with negligible cleavage) prior to administration. Split infusion: separate administration of individual bivalirudin components at peripheral sites on opposite sides of the body will allow central mixing and click ligation, activating the anticoagulant in the central circulation (e.g., super vena cava, right atrium, right ventricle). The rate of ultrafast TCO release controls the half-life of the clicked bivalirudin and its distribution in circulation prior to self-inactivation. Circuit infusion: analogous to central infusion, the click-bivalirudin is infused into the blooding entering a life-support circuit (e.g., extracorporeal membrane oxygenation (ECMO), ventricular assist device (VAD), or cardiopulmonary bypass circuit (bypass).

FIG. 2 contains synthetic scheme showing synthesis of P4-Tz.

FIG. 3 contains synthetic scheme showing synthesis of TCO-P12 and rTCO-P12.

FIG. 4A contains a synthetic scheme showing synthesis of ClickBival(TCO).

FIG. 4B contains a synthetic scheme showing synthesis of ClickBival(rTCO) FIG. 5A contains a synthetic scheme showing synthesis of iTCO-based P4 and P12 fragments.

FIG. 5B contains a scheme showing schematically showing ON/OFF control of biological activity of iTCO-based clickBival via click assembly and self-destruct cleavage.

FIG. 6A contains a line plot showing thrombin inhibition by ClickBival(TCO).

FIG. 6B contains a line plot showing thrombin inhibition by ClickBival(rTCO).

FIG. 7A contains LCMS chromatogram showing self-destruction of clickBival on clinically relevant timeframe.

FIG. 7B contains mass spectra showing formation of P4-DAP-PA2 and Gly2-P12 in the self-destruction reaction of clickbival.

FIG. 7C contains chemical structures of clickbival and corresponding fragments P4-DAP-PA2 and Gly2-P12.

FIG. 8A contains chemical structures of TCO-containing P4 and P12 fragments.

FIG. 8B contains chemical structures of TCO- and Tz-containing P4 and P12 fragments containing a linker X between the amino acid fragment and the reactive group.

### DETAILED DESCRIPTION

In one general aspect, the present application provides biologically active anticoagulant molecules containing trans-cyclooctene (“TCO”), tetrazine, and/or cycloocta[d]pyridazine moieties in their chemical structures. Due to the presence of these bioorthogonal fragments, the biologically active molecules can be disassembled “on demand” by tuning their self-destruction kinetics and therefore modulating their half-life.

### Compounds

In some embodiments, the present application provides a compound of Formula (I):

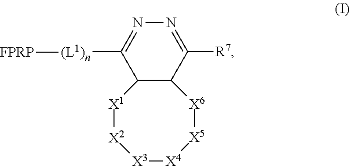

or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and a moiety of formula (i):

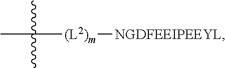

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound of Formula (I) comprises only one moiety of formula (i);
L¹ and L² are each independently selected from N(R^{N}), O, C(O), S, S(O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
m is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

In some embodiments, X¹ is O. In some embodiments, X¹ is CHR¹.

In some embodiments, X² is O. In some embodiments, X² is CHR².

In some embodiments, X³ is O. In some embodiments, X³ is CHR³.

In some embodiments, X⁴ is O. In some embodiments, X⁴ is CHR⁴.

In some embodiments, X⁵ is O. In some embodiments, X⁵ is CHR⁵.

In some embodiments, X⁶ is O. In some embodiments, X⁶ is CHR⁶.

In some embodiments, R¹, R², R³, R⁴, R⁵, and R⁶ are each independently selected from H, halo, C₁₋₆ alkyl, C₁₋₆ haloalkyl, C₁₋₃ alkoxy, and C₁₋₃ haloalkoxy.

In some embodiments, R¹, R², R³, R⁴, R⁵, and R⁶ are each independently selected from H, halo, and C₁₋₆ alkyl.

In some embodiments, each of the above pairs of R-groups, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, HO—C₁₋₃ alkylene, and NH₂—C₁₋₃ alkylene.

In some embodiments, each of the above pairs of R-groups, together with the carbon atoms to which they are attached, form an C₆₋₁₀ aryl ring (e.g., phenyl).

In some embodiments, each of the above pairs of R-groups, together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, each of the above pairs of R-groups, together with the carbon atoms to which they are attached, form a 5-14 membered heteroaryl ring (e.g., pyridinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, each of the above pairs of R-groups, together with the carbon atoms to which they are attached, form a 4-10 membered heterocycloalkyl ring (e.g., azetidinyl), which is optionally substituted with 1 or 2 substituents independently selected from C(O)OH, C(O)C₁₋₃ alkoxy, C₁₋₃ alkyl, HO—C₁₋₃ alkylene, and NH₂—C₁₋₃ alkylene.

In some embodiments, each L¹ is independently selected from N(R^{N}), O, C(═O), C₁₋₆ alkylene, —(OCH₂CH₂)ₓ—, —(CH₂CH₂O)ₓ—, an amino acid, a self-immolative group, and a moiety formed by a click reaction, each of which is optionally substituted with 1 or 2 substituents independently selected from C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy.

In some embodiments, each L¹ is independently selected from N(R^{N}), O, C(═O), C₁₋₆ alkylene, —(OCH₂CH₂)ₓ—, and —(CH₂CH₂O)ₓ—.

In some embodiments, at least one L¹ is N(R^{N}). In some embodiments, at least one L¹ is O. In some embodiments, at least one L¹ is C(═O). In some embodiments, at least one L¹ is C₁₋₆ alkylene, optionally substituted with C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy.

In some embodiments, n is an integer from 0 to 20. In some embodiments, n is an integer from 0 to 10. In some embodiments, n is 0, 1, 2, 3, 4, or 5.

In some embodiments, In some embodiments, each L² is independently selected from N(R^{N}), O, C(═O), C₁₋₆ alkylene, —(OCH₂CH₂)ₓ—, —(CH₂CH₂O)ₓ—, an amino acid, a self-immolative group, and a moiety formed by a click reaction, each of which is optionally substituted with 1 or 2 substituents independently selected from C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy.

In some embodiments, each L² is independently selected from N(R^{N}), O, C(═O), C₁₋₆ alkylene, —(OCH₂CH₂)ₓ—, and —(CH₂CH₂O)ₓ—.

In some embodiments, at least one L² is N(R^{N}). In some embodiments, at least one L² is O. In some embodiments, at least one L² is C(═O). In some embodiments, at least one L² is C₁₋₆ alkylene, optionally substituted with C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy.

In some embodiments, m is an integer from 0 to 20. In some embodiments, m is an integer from 0 to 10. In some embodiments, m is 0, 1, 2, 3, 4, or 5.

In some embodiments, each x is independently an integer from 1 to 2,000 (e.g., 10, 20, 100, 200, or 500).

In some embodiments, each R^{N} is independently selected from H, C₁₋₃ alkyl, and C₁₋₃ haloalkyl. In some embodiments, R^{N} is H. In some embodiments, R^{N} is C₁₋₃ alkyl.

### Amino Acids and Amino Acid Linker Moieties

Suitable examples of amino acids that could be used in L¹ or L² include lysine, arginine, histidine, aspartic acid, glutamic acid, serine, threonine, asparagine, glutamine, cysteine, glycine, proline, alanine, valine, isoleucine, leucine, methionine, phenylalanine, tyrosine, and tryptophan. Compounds and linker moieties with protected side chain functional groups are also encompassed by the term “amino acid” as used herein. A linker may include any n number of amino acids, for example, the linker can include (Gly)ₙ(aka Gₙ), where Gly can be N-linked or O-linked to any part of the remainder of the linker, and n can be an integer from 1 to 100 (e.g., 2, 3, 4, 5, 6, 7, 8, 9, or 10).

As used herein, the term “amino acid” generally refers to organic compounds containing amine (—NH₂) and carboxyl (—COOH) functional groups, along with a side chain (R group) specific to each amino acid. The side chain may be hydrophobic or hydrophilic, charged or neutral, as well as aliphatic or aromatic. In natural amino acids, the amine and carboxyl functional groups attached to the same carbon atom, i.e., an amino group is attached to the carbon in α-position relative to carboxyl group. Any of the amino acids described herein may be in L configuration or in D configuration. In some embodiments, the amino acid is in L configuration. In some embodiments, the amino acid is in D configuration. In one example, the amino acid F in the compounds of this disclosure is in D-configuration while the remaining amino acids are in L configuration. In another example, all amino acids in the compounds are in L configuration. The 20 natural amino acids are abbreviated herein as shown in Table A:

TABLE A

| Three-letter | One-letter | Amino |
|---|---|---|
| abbreviation | abbreviation | acid name |
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Ser | S | Serine |
| Thr | T | Threonine |
| Trp | W | Tryptophan |
| Tyr | Y | Tyrosine |
| Val | V | Valine |

### Self-Immolative Groups and Linker Moieties

Suitable examples of self-immolative groups include those described, for example, in Alouane, A. et al., “Self-immolative spacers: kinetic aspects, structure-property relationships, and applications”, Angew. Chem. Int. Ed., 2015, 54, 7492-7509 and Kolakowski, R. V. et al., “The methylene alkoxy carbamate self-immolative unit: Utilization of the targeted delivery of alcohol-containing payloads with antibody-drug conjugates”, Angew. Chem. Int. Ed., 2016, 55, 7948-7951.

Examples of self-immolative groups include the following groups of formulae (a)-(i):

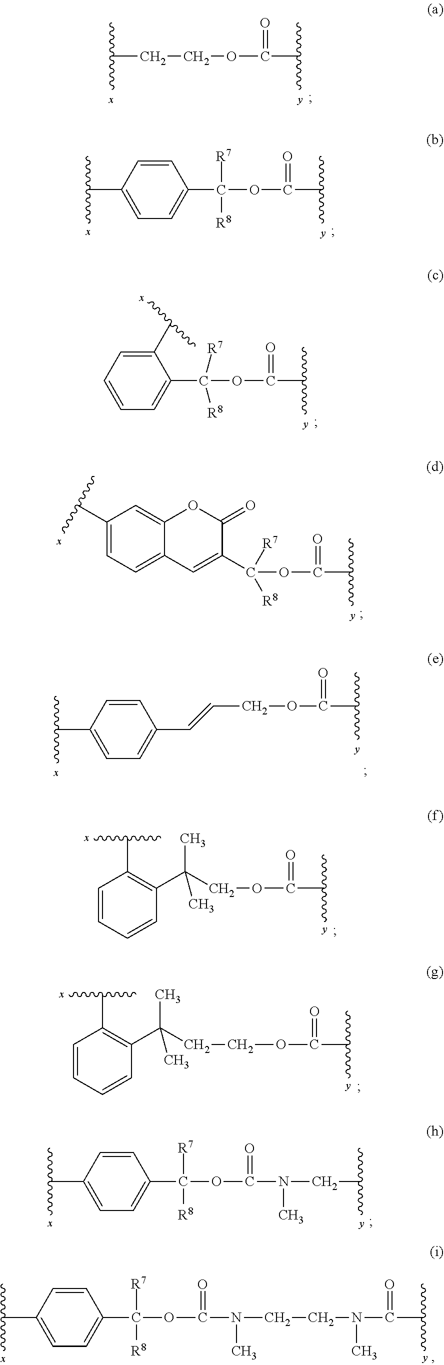

wherein x and y denote points of attachment to different L¹ or L², and R⁷ and R⁸ are each independently selected from H and C₁₋₆ alkyl.
   Groups and Moieties that are Formed by a “Click” Reaction

In some embodiments, the moiety formed by a click reaction is a reaction product of any one of the well-known click reactions, such as Huisgen cycloaddition (also known as [3+2]cycloaddition of alkynes and azides to form triazoles), Staudinger ligation (i.e., a reaction between an azide and a phosphine), a reaction of oxanorbornadienes and azides to from triazoles, an inverse-demand Diels-Alder reaction of tetrazines (e.g., dipyridyl tetrazines) and trans-cycloctynes, inverse-demand Diels-Alder reaction of tetrazines (e.g., monoaryl tetrazines) and norbornenes, a reaction of tetrazines and cyclopropenes, a reaction of cyclopropenes and nitrile imines, a photoinduced 1,3-dipolar cycloaddition of tetrazoles and alkenes, a 1,3-dipolar cycloaddition of nitrile oxides and norbornenes, a [4+1]cycloaddition isocyanides and tetrazines, or a 1,3-cycloaddition of nitrones and alkynes. In some embodiments, the moiety formed by a click reaction comprises a triazole. In some embodiments, the moiety formed by a click reaction is selected from:

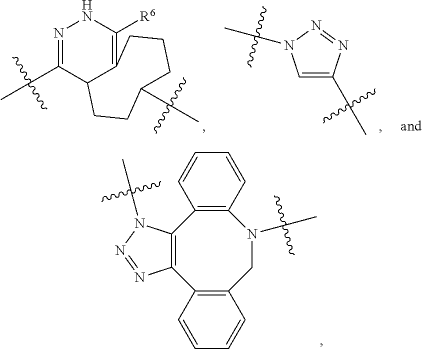

wherein R⁶ is selected from H and C₁₋₆ alkyl.

In some embodiments, the compound of Formula (I) has formula:

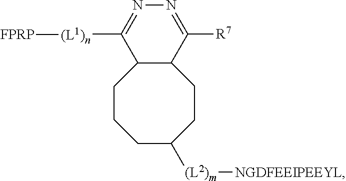

or a pharmaceutically acceptable salt thereof.

In some embodiments, the compound of Formula (I) has formula:

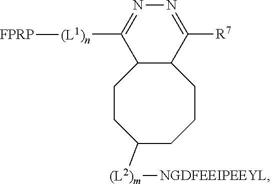

or a pharmaceutically acceptable salt thereof.

In some embodiments, the compound of Formula (I) has formula:

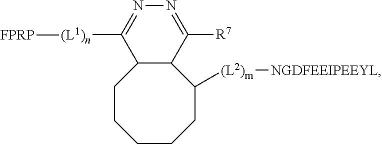

or a pharmaceutically acceptable salt thereof.

In some embodiments, the compound of Formula (I) has formula:

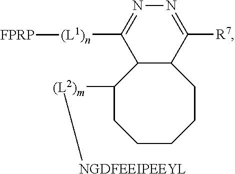

or a pharmaceutically acceptable salt thereof.

In some embodiments, the compound of Formula (I) has formula:

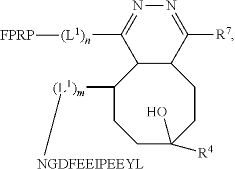

or a pharmaceutically acceptable salt thereof.

In some embodiments, the compound of Formula (I) has formula:

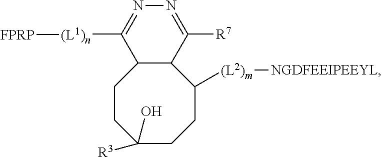

or a pharmaceutically acceptable salt thereof.

In some embodiments, L¹ comprises an amino acid. In some embodiments, L¹ comprises G₂, G₄, G₆, or G₈. In some embodiments, Licormrises —OC(═O)—, —NHC(═O)—, —C(═O)NH—, or —C(═O)O—. In some embodiments, L¹ comprises —N(R^{N})—C₁₋₆ alkylene-N(R^{N})—. In some embodiments, L¹ comprises —(OCH₂CH₂)ₓ— or —(CH₂CH₂O)ₓ—.

In some embodiments, L² comprises an amino acid. In some embodiments, L² comprises G₂, G₄, G₆, or G₈. In some embodiments, L² comprises —OC(═O)—, —NHC(═O)—, —C(═O)NH—, or —C(═O)O—. In some embodiments, L² comprises —N(R^{N})—C₁₋₆ alkylene-N(R^{N})—. In some embodiments, L² comprises —(OCH₂CH₂)ₓ— or —(CH₂CH₂O)ₓ—.

In some embodiments, R⁷ is C₁₋₆ alkyl, optionally substituted with OH, NH₂, or COOH. In some embodiments, R⁷ is C₁₋₆ alkyl. In some embodiments, R⁷ is C₆₋₁₀ aryl or 5-6-membered heteroaryl, each of which is optionally substituted with OH, NH₂, or C(O)OH. In some embodiments, R⁷ is C₂₋₄ alkenyl substituted with C₁₋₃ alkoxy. In some embodiments, R⁷ is 3,4-dihydro-2H-pyranyl.

In some embodiments, the compound of Formula (I) is selected from any one of the following compounds:

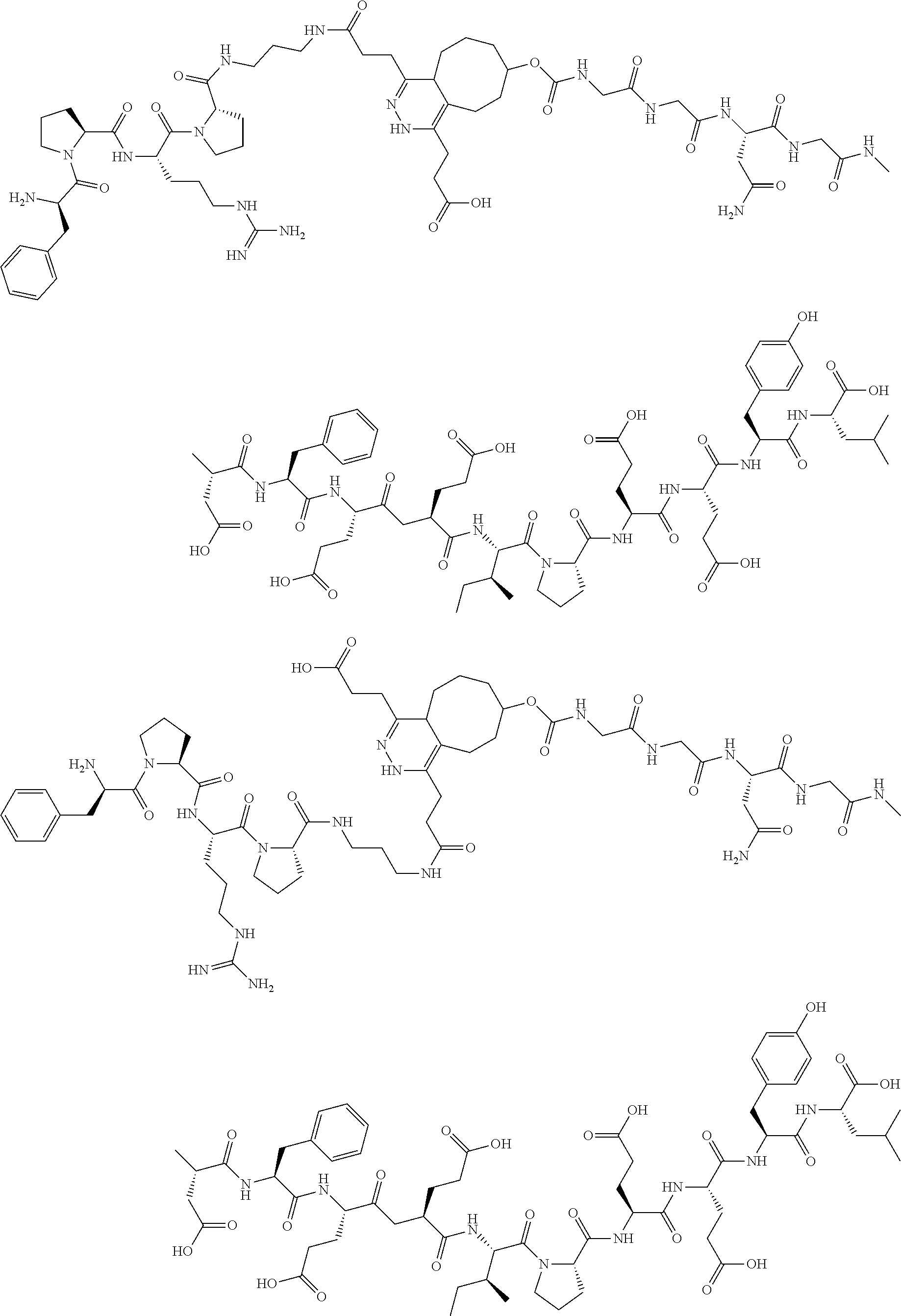

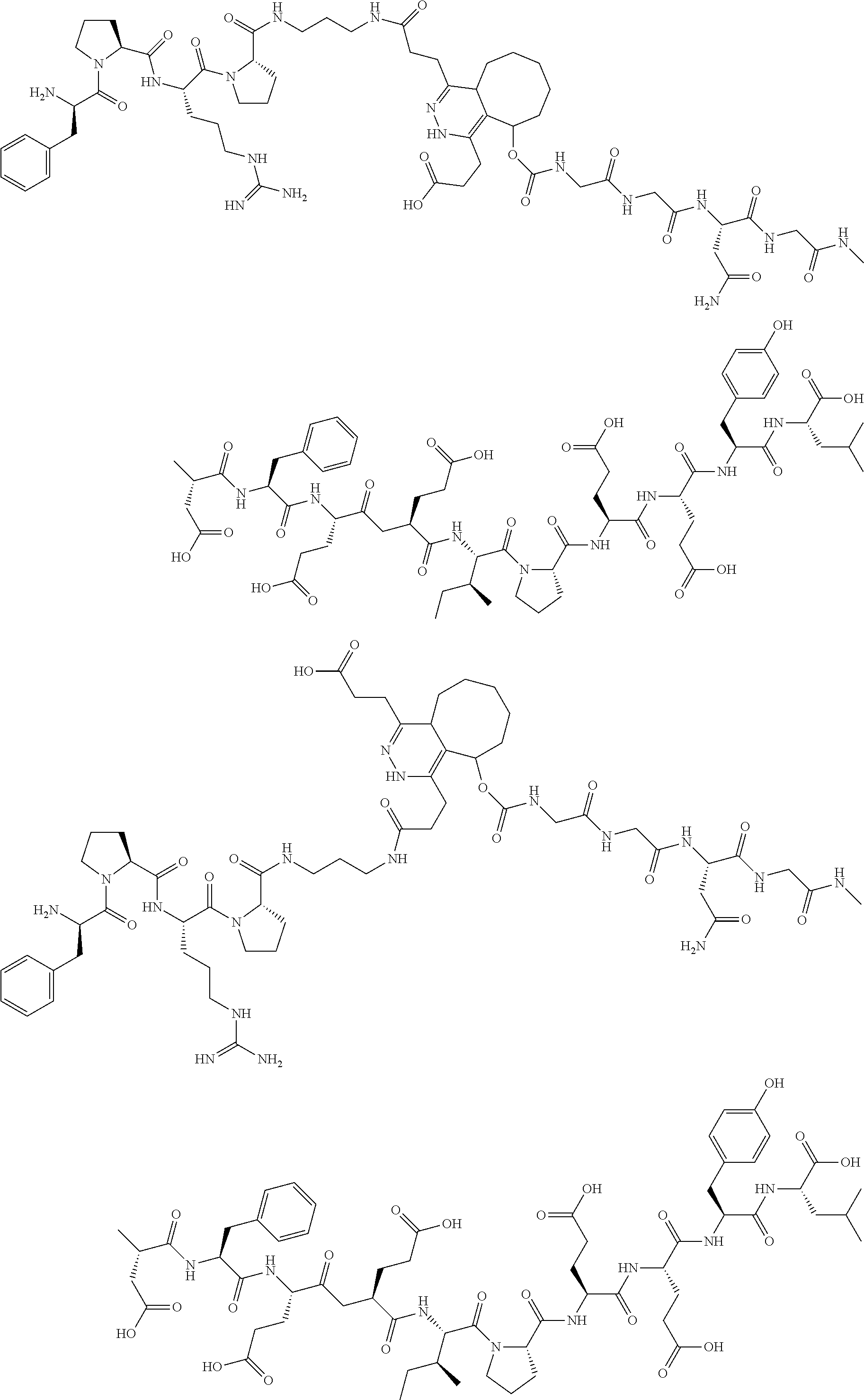

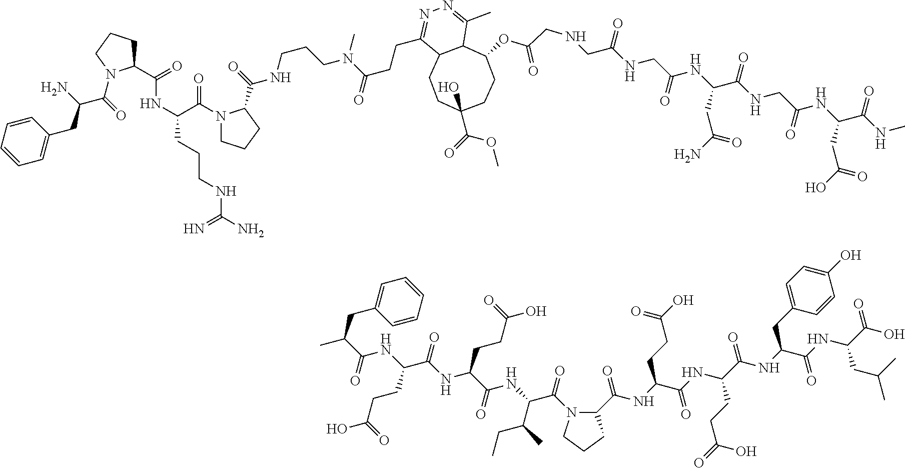

or a pharmaceutically acceptable salt thereof

In some embodiments, the present application provides a compound of Formula (II):

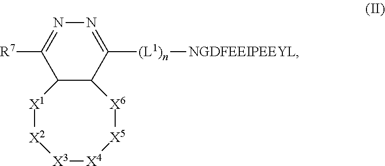

or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁴ is selected from O and C(R⁵)₂;
X⁶ is selected from O and C(R⁶)₂;
R¹, R², R³, R⁴, R⁵, and R⁶ are each independently selected from H, OH, N)₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and a moiety of formula (i):

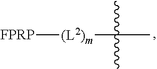

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound of Formula (I) comprises only one moiety of formula (i);
L¹ and L² are each independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
m is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

In some embodiments, the X¹—X⁶, R¹-R⁶, L¹, L², R⁷, n, m, x, R^{N}, amino acids, amino acid linkers, self-immolative groups and linkers, and groups and moieties that are formed by a “click” reaction, as described herein for Formula (I).

In some embodiments, the compound of Formula (II) has formula

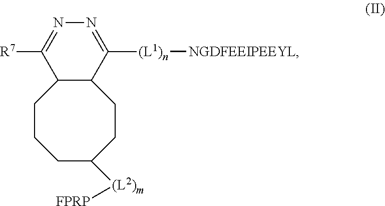

or a pharmaceutically acceptable salt thereof.

In some embodiments, the compound of Formula (II) has formula:

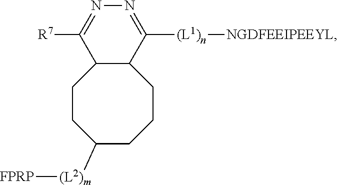

or a pharmaceutically acceptable salt thereof.

In some embodiments, the compound of Formula (II) has formula:

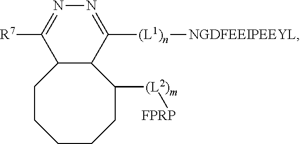

or a pharmaceutically acceptable salt thereof.

In some embodiments, the compound of Formula (II) has formula:

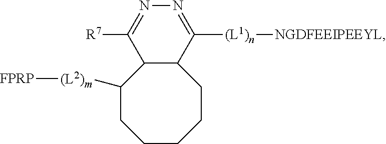

or a pharmaceutically acceptable salt thereof.

In some embodiments, the compound of Formula (II) has formula:

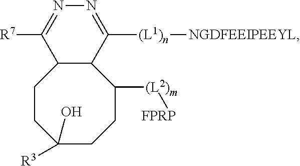

or a pharmaceutically acceptable salt thereof.

In some embodiments, the compound of Formula (II) has formula:

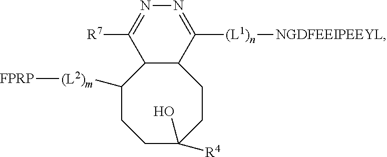

or a pharmaceutically acceptable salt thereof.

In some embodiments, the compound of Formula (II) is selected from any one of the following compounds:

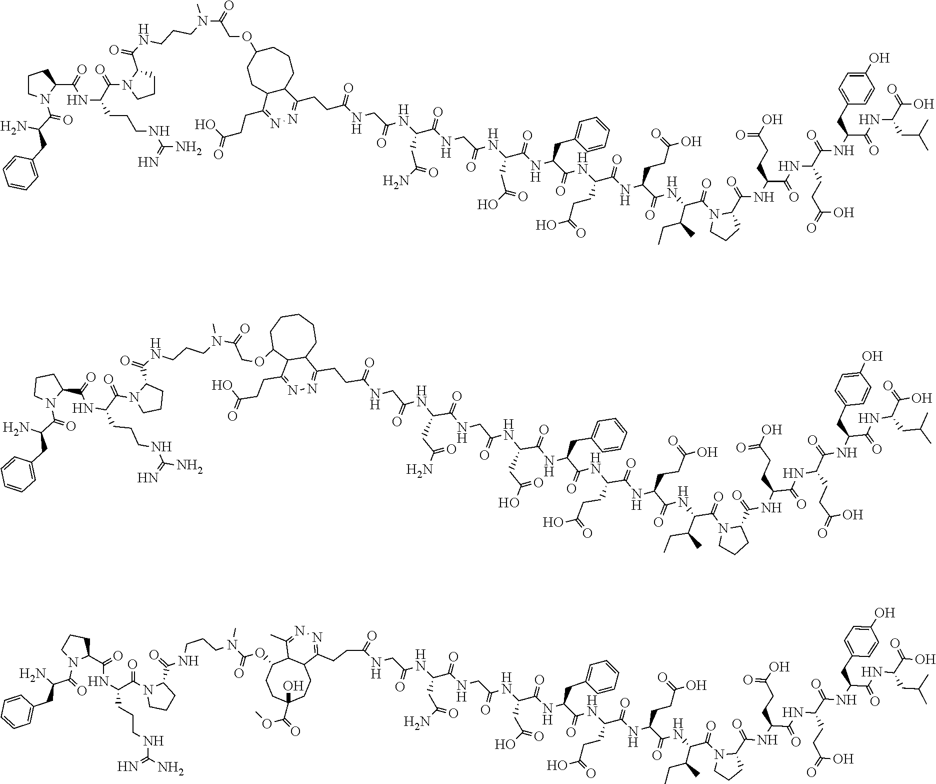

or a pharmaceutically acceptable salt thereof.

In some embodiments, the present disclosure provides a compound of Formula (III):

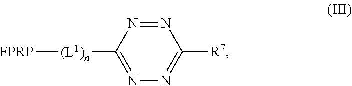

or a pharmaceutically acceptable salt thereof, wherein:
each L¹ is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

In some embodiments, L¹, R^{N}, x, n, and R⁷ are as described herein for Formula (I).

In some embodiments, (L¹)ₙ comprises —(OCH₂CH₂)ₓ—, —(CH₂CH₂O)ₓ—, C₁₋₃ alkylene, C(═O), (amino acid)ₙ, or —N(R^{N})—C₁₋₃ alkylene-N(R^{N})—.

In some embodiments, n is selected from 1, 2, 3, 4, or 5.

In some embodiments, R⁷ is C₁₋₆ alkyl, optionally substituted with OH, NH₂, or COOH.

In some embodiments, R⁷ is C₁₋₆ alkyl.

In some embodiments, R⁷ is C₆₋₁₀ aryl or 5-6-membered heteroaryl, each of which is optionally substituted with OH, NH₂, or C(O)OH. In some embodiments, R⁷ is C₂₋₄ alkenyl substituted with C₁₋₃ alkoxy, or R⁷ is a pyranyl.

In some embodiments the compound of Formula (II) is selected from:

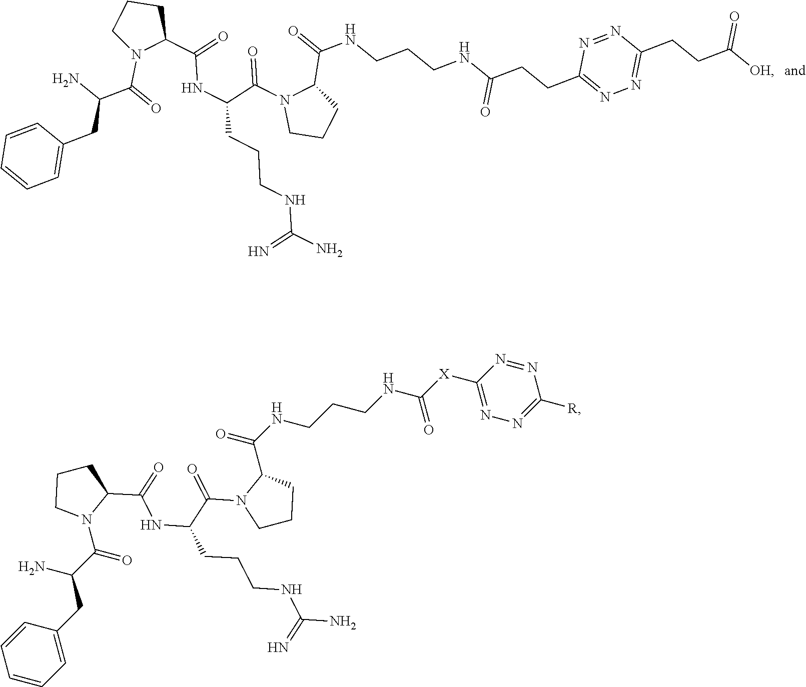

wherein X is selected from methylene, ethylene, and propylene, and R is selected from methyl, 2-carboxyethyl, and 2-aminoethyl.
or a pharmaceutically acceptable salt thereof. In some embodiments, X is ethylene. In some embodiments, R is methyl. In some embodiments, R is 2-carboxyethyl.

In some embodiments, the present disclosure provides a compound of Formula (IV):

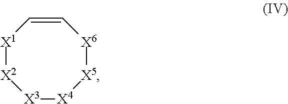

or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and a moiety of formula (i):

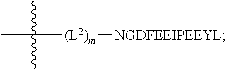

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound of Formula (IV) comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl.

In some embodiments, the X¹—X⁶, R¹-R⁶, L¹, L², R⁷, n, m, x, R^{N}, amino acids, amino acid linkers, self-immolative groups and linkers, and groups and moieties that are formed by a “click” reaction, as described herein for Formula (I).

In some embodiments, L² comprises —OC(═O)—, —NHC(═O)—, —C(═O)NH—, or —C(═O)O—. In some embodiments, L² comprises —N(R^{N})—C₁₋₆ alkylene-N(R^{N})—. In some embodiments, L² comprises —(OCH₂CH₂)ₓ— or —(CH₂CH₂O)ₓ—. In some embodiments, m is selected from 1, 2, 3, 4, and 5.

In some embodiments, the compound of Formula (IV) has formula:

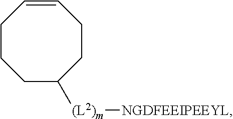

or a pharmaceutically acceptable salt thereof.

In some embodiments, the compound of Formula (IV) has formula:

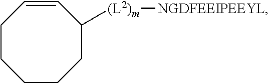

or a pharmaceutically acceptable salt thereof.

In some embodiments, the compound of Formula (IV) has formula:

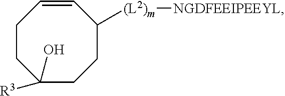

or a pharmaceutically acceptable salt thereof.

In some embodiments, the compound of Formula (IV) is selected from any one of the following compounds:

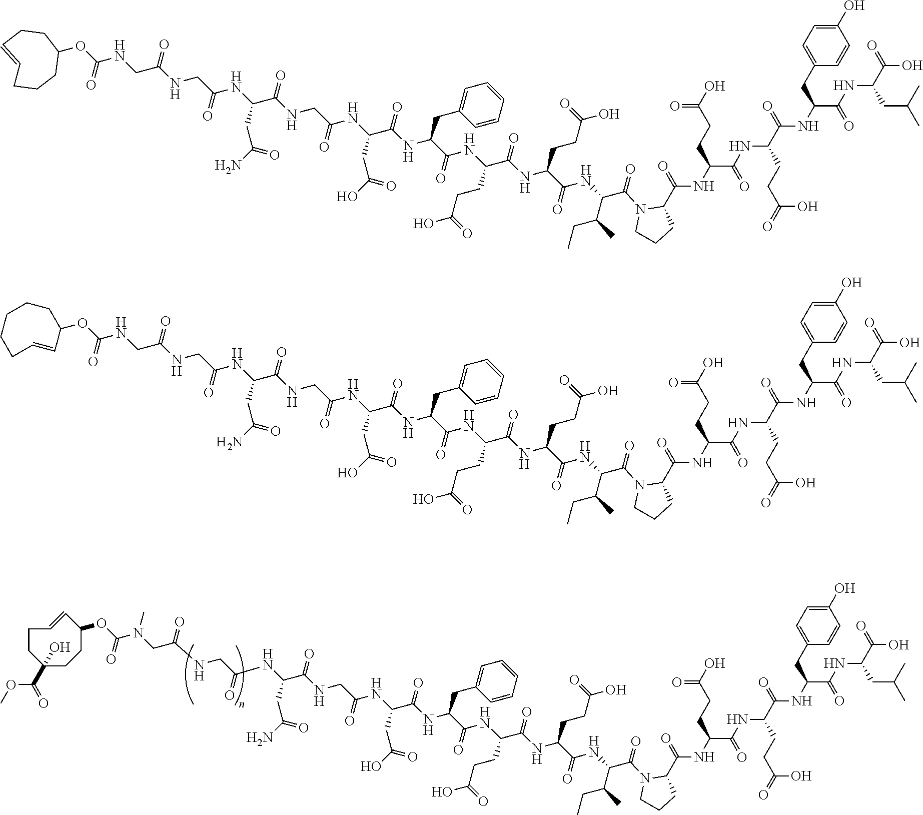

or a pharmaceutically acceptable salt thereof.

In some embodiments, the present disclosure provides a compound of Formula (V).

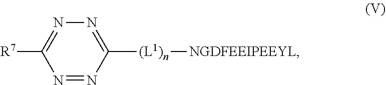

or a pharmaceutically acceptable salt thereof, wherein:
each L¹ is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

In some embodiments, L¹, R^{N}, x, n, and R⁷ are as described herein for Formula (I).

In some embodiments, (L¹)ₙ comprises —(OCH₂CH₂)ₓ—, —(CH₂CH₂O)ₓ—, C₁₋₃ alkylene, C(═O), (amino acid)ₙ, or —N(R^{N})—C₁₋₃ alkylene-N(R^{N})—. In some embodiments, n is selected from 1, 2, 3, 4, or 5. In some embodiments, R⁷ is C₁₋₆ alkyl, optionally substituted with OH, NH₂, or COOH. In some embodiments, R⁷ is C₁₋₆ alkyl. In some embodiments, R⁷ is C₆₋₁₀ aryl or 5-6-membered heteroaryl, each of which is optionally substituted with OH, NH₂, or C(O)OH.

In some embodiments, the compound of Formula (V) is selected from any one of the following compounds:

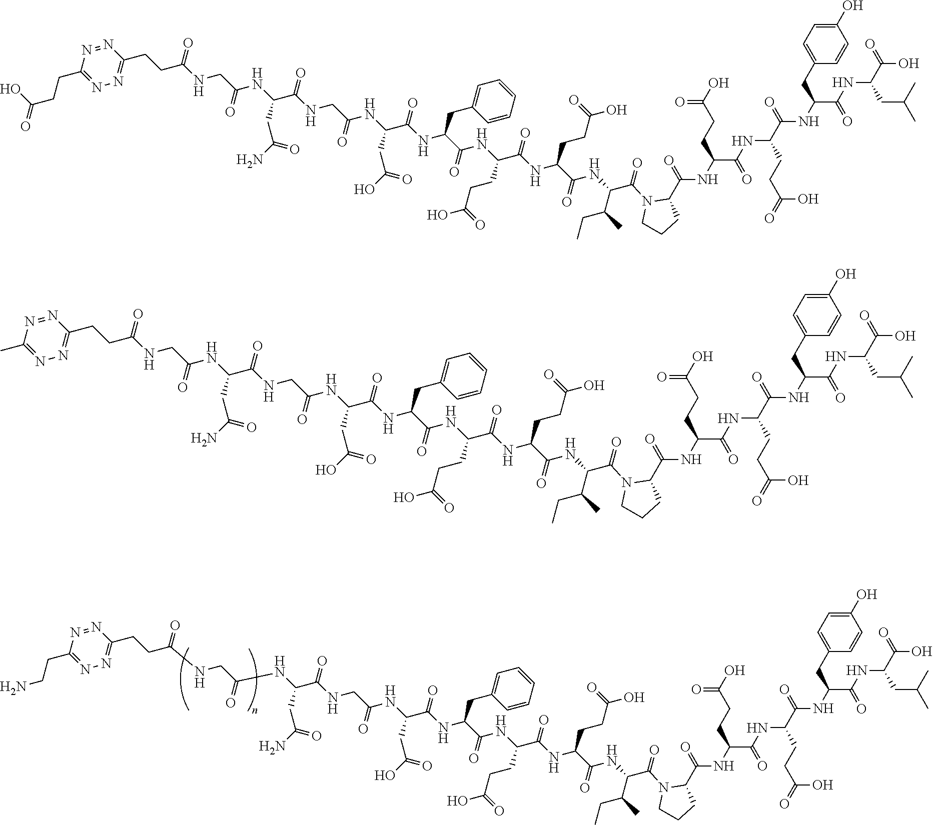

wherein n is 2, 4, or 6,
or a pharmaceutically acceptable salt thereof. In some embodiments, n is 2. In some embodiments, n is 4. In some embodiments, n is 6.

In some embodiments, the present disclosure provides a compound of Formula

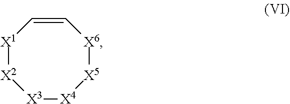

or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and a moiety of formula (i):

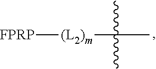

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound of Formula (VI) comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl.

In some embodiments, the X¹—X⁶, R¹, R⁶, L², m, x, R^{N}, amino acids, amino acid linkers, self-immolative groups and linkers, and groups and moieties that are formed by a “click” reaction, are as described herein for Formula (I).

In some embodiments, L² comprises —OC(═O)—, —NHC(═O)—, —C(═O)NH—, or —C(═O)O—. In some embodiments, L² comprises —N(R^{N})—C₁₋₆ alkylene-N(R^{N})—. In some embodiments, L² comprises —(OCH₂CH₂)ₓ— or —(CH₂CH₂O)ₓ—. In some embodiments, m is selected from 1, 2, 3, 4, and 5.

In some embodiments, the compound of Formula (VI) has formula:

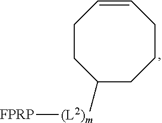

or a pharmaceutically acceptable salt thereof.

In some embodiments, the compound of Formula (VI) has formula:

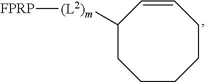

or a pharmaceutically acceptable salt thereof.

In some embodiments, the compound of Formula (VI) has formula:

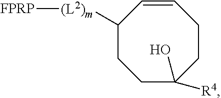

or a pharmaceutically acceptable salt thereof.

In some embodiments, the compound of Formula (VI) is selected from any one of the following compounds:

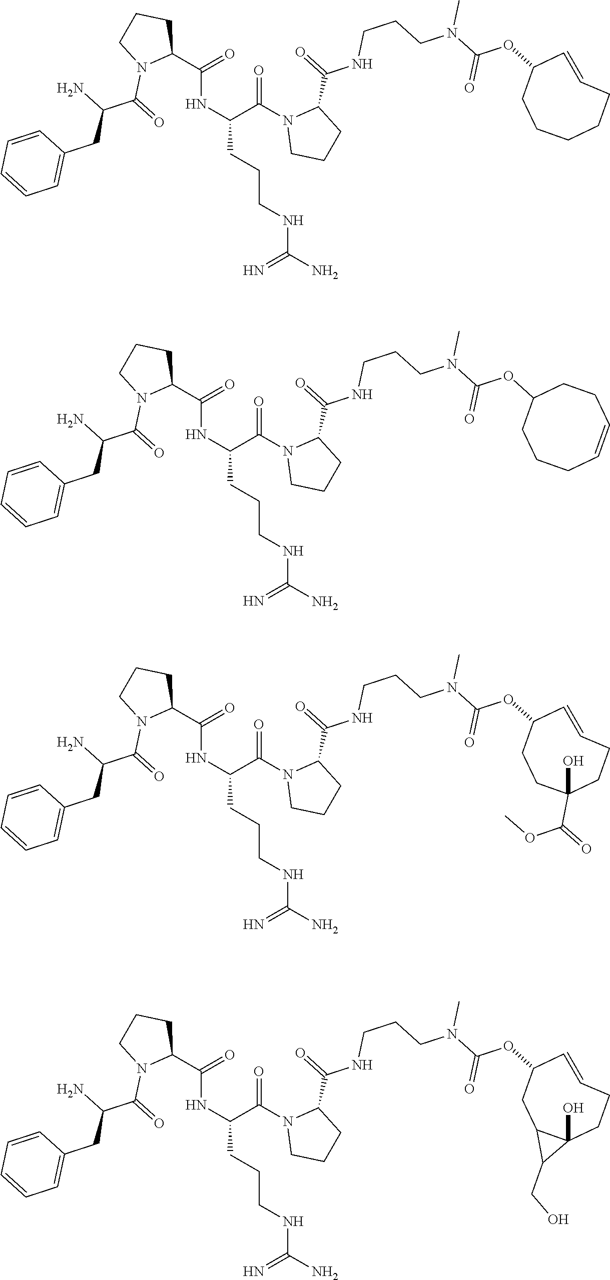

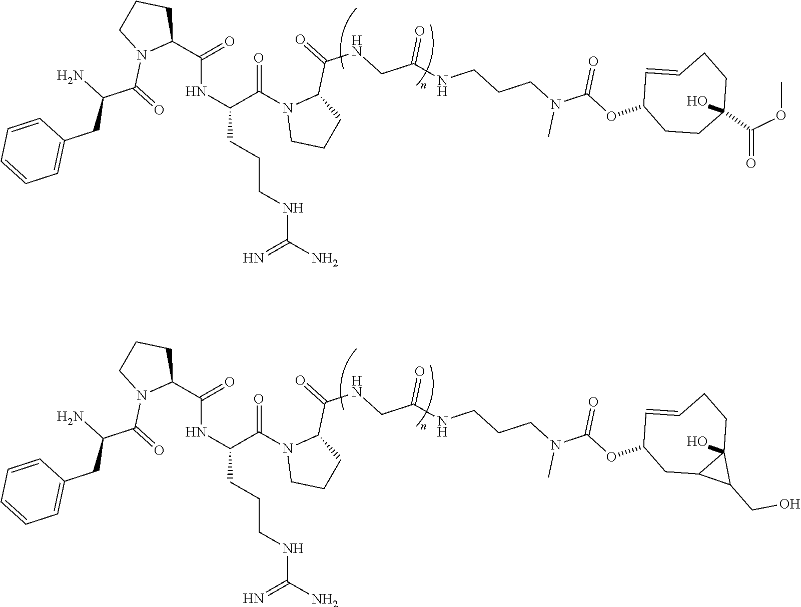

wherein n is 2, 4, or 6,
or a pharmaceutically acceptable salt thereof. In some embodiments, n is 2. In some embodiments, n is 4. In some embodiments, n is 6.

### Salts and Pharmaceutically Acceptable Salts

In some embodiments, a salt (such as a pharmaceutically acceptable salt) of a compound of the present disclosure is formed between an acid and a basic group of the compound, such as an amino functional group, or a base and an acidic group of the compound, such as a carboxyl functional group. According to another embodiment, the compound is an acid addition salt (e.g., a pharmaceutically acceptable addition salt)

In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Methods of Making of the Compounds

In some embodiments, the present disclosure provides a method of making a compound of Formula (I) as described herein, the method comprising reacting a compound of Formula (III), or a pharmaceutically acceptable salt thereof, as described herein, with a compound of formula (IV), or a pharmaceutically acceptable salt thereof, as described herein, to obtain the compound of Formula (I), or a pharmaceutically acceptable salt thereof.

In some embodiments, the present disclosure provides a method of making a compound of Formula (II), or a pharmaceutically acceptable salt thereof, as described herein, the method comprising reacting a compound of Formula (V), or a pharmaceutically acceptable salt thereof, as described herein, with a compound of formula (VI), or a pharmaceutically acceptable salt thereof, as described herein, to obtain the compound of Formula (II), or a pharmaceutically acceptable salt thereof.

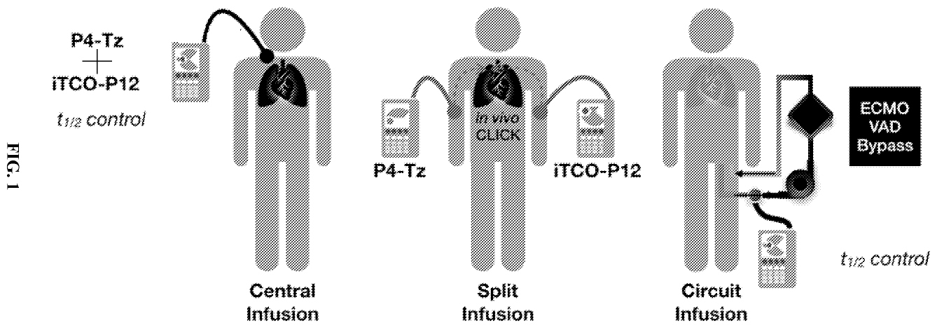

In some embodiments, the reacting is carried out in a physiologically acceptable medium. In some embodiments, the physiologically acceptable medium comprises a buffer comprising pH from about 5.5 to about 8, a saline, a dextrose solution, or an aqueous solution suitable for injection or infusion. In some embodiments, the reacting is carried out at about room temperature. The reacting time can be 1 sec, 2 sec, 5 sec, 1 min, 2 min, 4 min, 5 min, or 10 min. The compounds of Formula (I) or (II) can be stored at reduced temperature (e.g., −80° C.), or can be prepared on site of administration and administered to the subject upon making. The compounds can be administered to by infusing the compound into the blood intake circuit of a life support device, as shown in FIG. 1, for example.

The compounds of Formulae (III), (IV), (V), and (VI) ca be obtained from P4 and P12 fragments of bivalirudin, suing suitable tetrazine and TCO reagents, as well as suitable liner reagents, using organic chemistry techniques and protecting groups as is commonly known in organic synthesis. A skilled organic chemist would be able to select and implement appropriate routes and protocols.

### Methods of Use

Anticoagulation (“AC”)—the management and prevention of blood clotting—is a foundational element of modern medical care, a cardinal issue in neurology, cardiology, and surgery, and relevant to every hospitalized patient. In spite of ongoing development of new therapeutic agents/antidotes, two major perennial challenges remain: i) medications that modulate coagulation invariably increase the risk of bleeding, creating a narrow therapeutic window; ii) patients often suffer paradoxically from simultaneous clotting and bleeding, creating irreconcilable dilemmas in which enhanced anticoagulation is needed in one physiologic territory/circuit and enhanced hemostasis in another.

In this context there are multiple high-acuity scenarios in which no safe approach exists, e.g., i) clinically significant pulmonary embolism juxtaposed with GI or CNS bleeding; ii) surgery in patients with a compelling need for ongoing anticoagulation or acute venous thrombosis after an operation; iii) obligatory anticoagulation for mechanical life support devices in patients with concurrent bleeding. While catheter-directed approaches enable semi-selective local delivery of fibrinolytics to treat life-threatening clots in a subset of these scenarios, they nonetheless confer a significant systemic bleeding risk. There is no corresponding approach for locoregional therapeutic AC; systemic doses can be escalated carefully, but binary decision-making is ultimately required.

The biologically active compounds and methods of the present disclosure advantageously allow to spatially restrict the scope of anticoagulation to affected regions within the circulation. Examples of such use include selective (i) cardiopulmonary AC for pulmonary embolism, atrial fibrillation, or mechanical heart valve prophylaxis, sparing hemostasis at peripheral loci of active bleeding/risk; (ii) in the surgical context, a ability to modulate AC selectively at the operative site; and (iii) selective extra-corporeal AC within a life-support circuit (e.g., ECMO, VAD, cardiopulmonary bypass) that requires AC even when the patient does not, minimizing hemostatic impact on the patient.

Given continuous recirculation in the blood stream, the half-lives of conventional anticoagulants are far too long to achieve selective local or locoregional delivery downstream from the site of infusion. Therapeutic half-life of such anticoagulants remains dependent on the complex and molecularly extrinsic interplay of biodistribution, metabolism, and excretion.

For example, bivalirudin is thrombin inhibitor and an anticoagulant indicated in patients with unstable angina undergoing percutaneous transluminal coronary angioplasty (PTCA), and indicated for use as an anticoagulant in patients undergoing percutaneous coronary intervention (PCI), including patients with ST-segment elevation myocardial infarction (STEMI) undergoing primary PCI. Bivalirudin is also indicated for the treatment of adult patients with unstable angina/non-ST segment elevation myocardial infarction (UA/NSTEMI) planned for urgent or early intervention. Bivalirudin can be co-administered with aspirin and/or clopidogrel. Bivalirudin is a 20 amino acid long peptide with the sequence D-Phe-Pro-Arg-Pro-Gly-Gly-Gly-Gly-Asn-Gly-Asp-Phe-Glu-Glu-Ile-Pro-Glu-Glu-Tyr-Leu (FPRPGGGGNGDFEEIPEEYL), where the first residue is D-phenylalanine instead of the natural L-phenylalanine. Bivalirudin directly inhibits thrombin by specifically binding both to the catalytic site and to the anion-binding exosite of circulating and clot-bound thrombin. Half-life of bival is about 20 min. Advantageously, the compounds of this disclosure provide bival half-life from about 5 sec to about 10 min, from about 10 sec to about 10 min, or from about 5 sec to about 5 min, such as about sec, about 10 sec, about 20 sec, about 30 sec, about 1 min, about 2 min, about 3 min, about 5 min, about 6 min, about 10 min, or about 15 min.

The approach shows broad utility in selective delivery of therapies to specific vascular beds, tissues, or tumors, and allows for management of bleeding and thrombosis and therefore effective regulation of anticoagulation.

Including bioorthogonal moiety within the chemical structure of a biologically active compound allows for spatiotemporal control to localize activity or mitigate systemic toxicity. This is accomplished by inactivating the biologically active compound using a bioorthogonal self-destructing chemistry systemically at a desired time or locally at a tissue within the patient's body where the biological activity of the compound is no longer desired, as described herein.

In some embodiments, the present disclosure provides a method of treating or preventing coagulation and/or blood clotting in a subject in need thereof, the method comprising administering to the subject a therapeutically effective amount of a compound of formula (I) or formula (II), or a pharmaceutically acceptable salt thereof, or a pharmaceutical composition comprising same.

In some embodiments, the subject in need thereof has unstable angina undergoing percutaneous transluminal coronary angioplasty.

In some embodiments, the subject in need thereof undergoes percutaneous coronary intervention.

In some embodiments, the present disclosure provides a method of treating or preventing coagulation and/or blood clotting in a subject in need thereof, the method comprising administering to the subject a therapeutically effective amount of a compound of Formula (III), or a pharmaceutically acceptable salt thereof, or a pharmaceutical composition of same, in combination with a therapeutically effective amount of a compound of Formula (IV), or a pharmaceutically acceptable salt thereof, or a pharmaceutical composition of same.

In some embodiments, the subject in need thereof has unstable angina undergoing percutaneous transluminal coronary angioplasty.

In some embodiments, the subject in need thereof undergoes percutaneous coronary intervention.

In some embodiments, the method includes administering the compound of Formula (III), or a pharmaceutically acceptable salt thereof, and the compound of Formula (IV), or a pharmaceutically acceptable salt thereof, in about stoichiometric amounts.

In some embodiments, the present disclosure provides a method of treating or preventing coagulation and/or blood clotting in a subject in need thereof, the method comprising administering to the subject a therapeutically effective amount of a compound of Formula (V), or a pharmaceutically acceptable salt thereof, or a pharmaceutical composition of same, in combination with a therapeutically effective amount of a compound of Formula (VI), or a pharmaceutically acceptable salt thereof, or a pharmaceutical composition of same.

In some embodiments, the subject in need thereof has unstable angina undergoing percutaneous transluminal coronary angioplasty.

In some embodiments, the subject in need thereof undergoes percutaneous coronary intervention.

In some embodiments, the method comprises administering the compound of Formula (V), or a pharmaceutically acceptable salt thereof, and the compound of Formula (VI), or a pharmaceutically acceptable salt thereof, in about stoichiometric amounts.

### Compositions, Formulations, and Routes of Administration

In some embodiments, the present disclosure provides a composition comprising a compound or a salt thereof (e.g., a pharmaceutically acceptable salt thereof), and an inert carrier (e.g., a pharmaceutically acceptable carrier). In some embodiments, the composition is an aqueous solution (i.e., the inert carrier is water). The aqueous solution may be a buffer, such as any buffer containing inert carrier such as water, phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, or any combination thereof. Some examples of buffers include Dulbecco's phosphate-buffered saline (DPBS), phosphate buffered saline, and Krebs-Henseleit Buffer. The pH of the buffer may be from about 5 to about 9, for example pH may be 6-8. In one example, the compound, or a salt thereof, containing a reactive group, may be admixed with the protein (e.g., antibody) or a small-molecule drug in any of the aqueous solutions described here to obtain a conjugate or a biologically active molecule containing a TCO fragment as described herein.

A composition (e.g., an aqueous solution) comprising the compound or conjugate of the present disclosure, may be used to treat a cell (e.g., a cell containing a biomarker) to image the cell using a fluorophore in the compound or conjugate.

The present application also provides pharmaceutical compositions comprising an effective amount of a compound, a conjugate, or a biologically active molecule of the present disclosure, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compound, conjugate, biologically active molecule, or therapeutic agent disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein. In compositions can also be administered on a tip of a catheter.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, the compound, conjugate, biologically active molecule is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound, conjugate, or biologically active molecule can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cn-m” indicates a range which includes the endpoints, wherein n and m are integers and indicate the number of carbons. Examples include C₁₋₄, C₁₋₆, and the like.

As used herein, the term “Cn-m alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cn-m haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cn-m alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cn-m alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cn-m haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cn-m alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cn-m-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “carboxy” refers to a —C(O)OH group.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cn-m aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl. The term “arylene” refers to a divalent aryl group, such as a phenylene.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl. The term “cycloalkylene” refers to a divalent cycloalkyl group, such as cyclopropylene.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C=N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

### EXAMPLES

### Example 1—Synthesis of P4-Tz

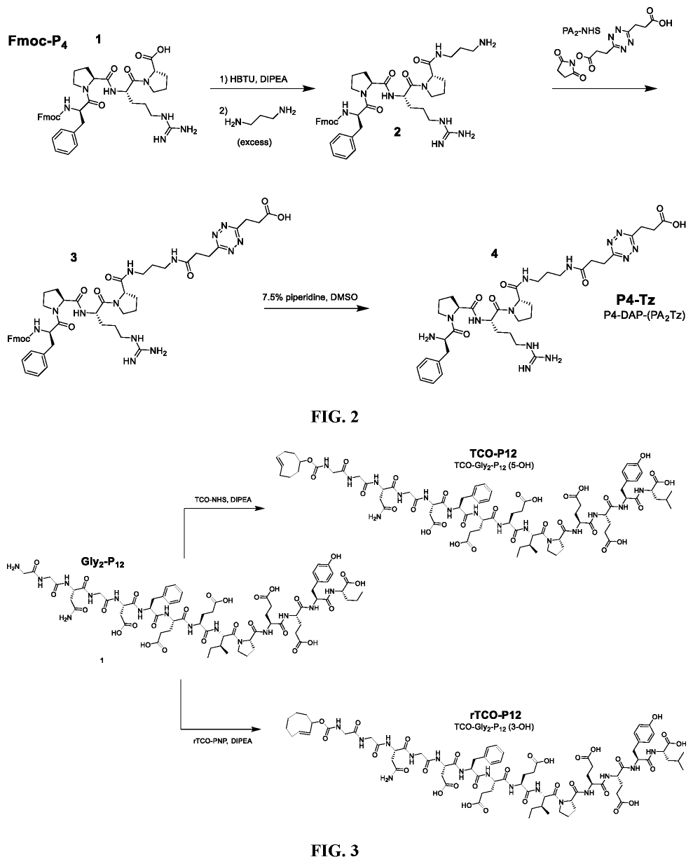

Synthetic scheme is shown in FIG. 2.

Compound 1: Fmoc-P4 (1) (Fmoc-(N-terminal)-{D-Phe}PRP, MW 737.86) was purchased as a custom-synthesized peptide from Genscript (New Jersey, USA) at >98% purity and used as supplied.

Compound 2: To solution of 1 in dry DMSO were added HBTU (1.1 equivalents) and DIPEA (2 equivalents). The reaction was allowed to proceed for 2 hours at room temperature, whereupon diaminopropane was added in excess (5 equivalents). After a further 15 minutes of reaction time, the reaction was neutralized by the addition of 5 equivalents of formic acid and then purified by reversed phase column chromatography (H2O/MeCN gradient elution).

Compound 3: To a solution of 2 in dry DMSO were added DIPEA (2 equivalents) and PA2-NHS (2 equivalents, prepared as in Carlson et al, JACS 2018). After 10 minutes, LCMS indicated that the reaction was complete, whereupon the product was purified by reversed phase chromatography (H2O/MeCN gradient).

Compound 4: Compound 3 was dissolved in dry DMSO, followed by the addition of neat piperidine (7.5% v/v). LCMS monitoring at t=5 minutes revealed complete cleavage of the Fmoc group. The reaction mix was directly injected onto a C₁₈ reverse phase column prior to precipitation of the cleaved Fmoc byproducts and purified by buffered gradient elution (ammonium formate, pH 8.5, MeCN) to yield P4-Tz (4). ESI-MS [M+H+]m/z calcd. 780.42 for C₃₆H54N13O7, found 780.82.

### Example 2—Synthesis of TCO-P12 and rTCO-P12

Synthetic scheme is shown in FIG. 3.

Compound 1: Gly₂-P₁₂ (GGNGDFEEIPEEYL, MW 1554.59) was purchased as a custom-synthesized peptide from Genscript (New Jersey, USA) at >98% purity and used as supplied.

TCO-P₁₂: To a solution of Gly₂P₁₂ (1) in dry DMSO were added 1-3 equivalents of TCO-NHS (Click Chemistry Tools, Arizona, USA) and 2 equivalents of DIPEA. After one hour, LCMS indicated complete acylation of the peptide starting material. The reaction mixture was then loaded onto a Biotage SNAP C₁₈Bio (20 μm spherical silica, 300 Å pore size) reverse phase column and purified by gradient elution (H2O:MeCN, 5%-60%).

rTCO-P₁₂: To a solution of Gly₂P₁₂ (1) in dry DMSO were added 1-3 equivalents of rTCO-PNP (prepared as in Versteegen et al, Angew Chem 2013) and 2 equivalents of DIPEA. Evolution of a yellow color was immediately noted. After 25 minutes, reaction monitoring by LCMS indicated complete labeling. The reaction mixture was directly injected onto a Biotage SNAP C₁₈Bio (20 μm spherical silica, 300 Å pore size) reverse phase column and purified by buffered gradient elution (ammonium formate, pH 9.2:MeCN, 5% until yellow p-nitrophenol fully eluted, followed by 5-60% MeCN ramp).

### Example 3—Synthesis of ClickBival

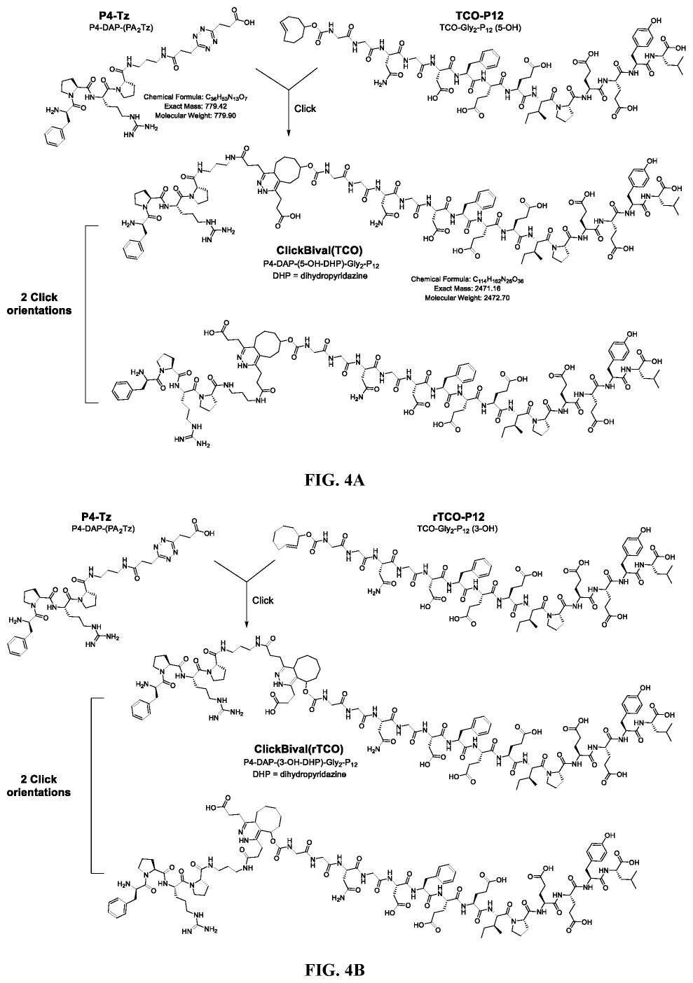

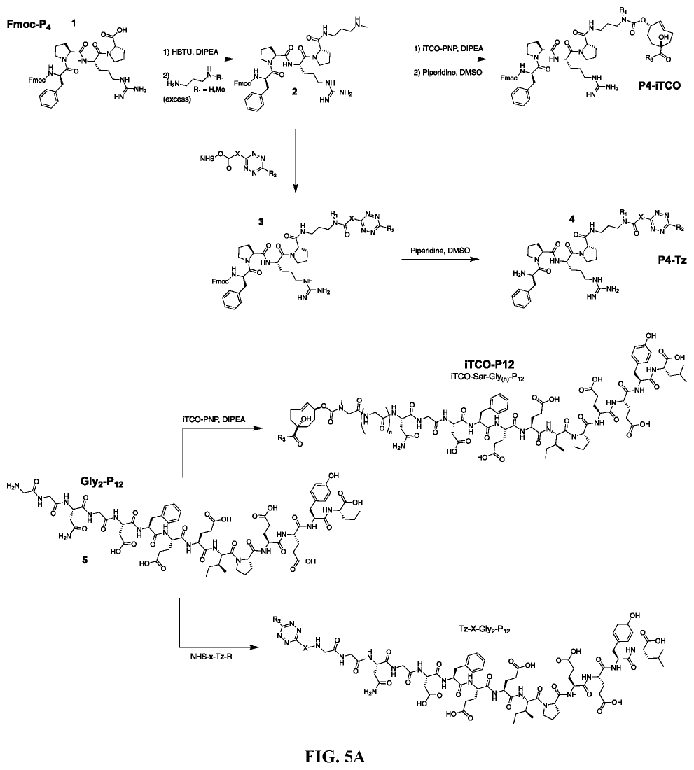

Synthetic scheme for TCO compound is shown in FIG. 4A.

Immediately prior to enzyme testing, DMSO stock solutions of P4-Tz (15.5 mM) and TCO-P12 (TCO-Gly2-P12 (5-OH), 14.5 mM) were combined in an Eppendorf tube, mixed thoroughly by pipette/vortex, and allowed to react for 1-5 minutes before addition of an aliquot of the reaction mixture (1-2 μL) to the enzyme-substrate reaction in the fluorimeter.

Synthetic scheme for rTCO compound is shown in FIG. 4B.

Immediately prior to enzyme testing, DMSO stock solutions of P4-Tz (15.5 mM) and TCO-P12 (rTCO-Gly2-P12 (3-OH), 14.5 mM) were combined in an Eppendorf tube, mixed thoroughly by pipette/vortex, and allowed to react for 1-5 minutes. An aliquot of the reaction mixture (1-2 μL) was then added directly to the enzyme-substrate reaction in the fluorimeter cuvette.

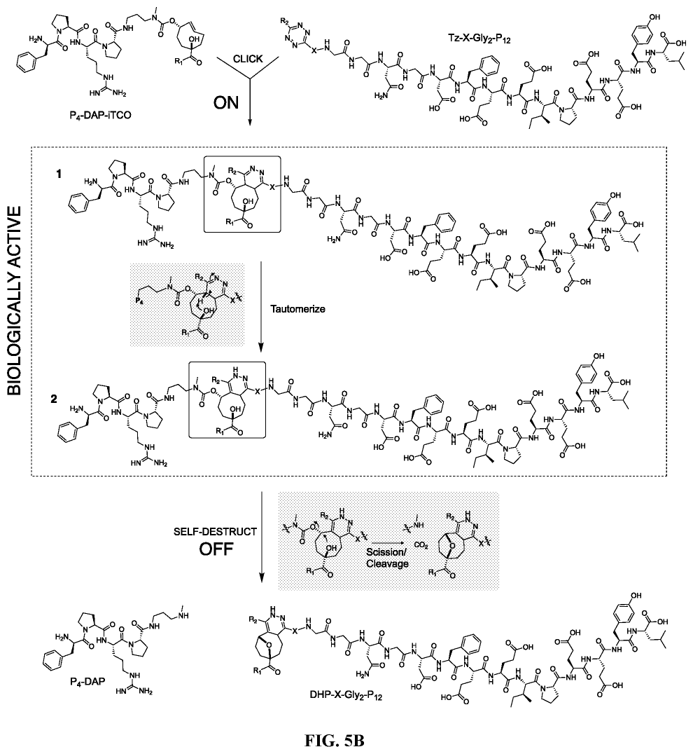

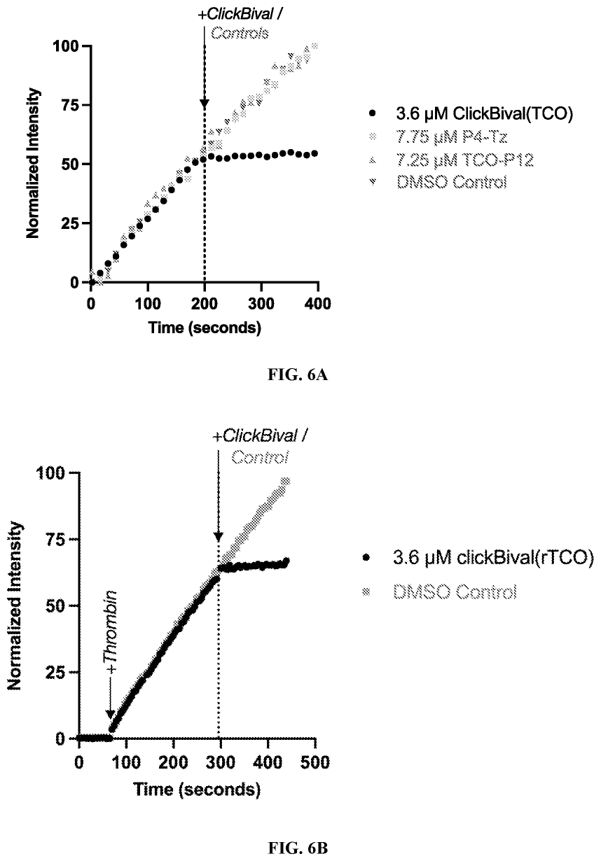

Syntheses of iTCO-based P4 and P12 fragments are shown in FIG. 5A. FIG. 5B shows ON/OFF control of biological activity via click assembly and self-destruct cleavage. The individual P4 and P12 components are biologically inactive until they click (“ON”) to assemble the biologically active species 1. This remains an active anticoagulant while undergoing the tautomerization reaction to form 2. After a time-limited period of activity, the intramolecular scission reaction cleaves the chain, and the self-destruct sequence (“OFF”) is complete, producing the separate P4 and P12 components and terminating biological activity.

### Example 4—Thrombin Inhibition by ClickBival

Thrombin inhibition by ClickBival(TCO) is shown in FIG. 6A. The ability of click-assembled bivalirudin (ClickBival) to inhibit human thrombin was tested in a fluorogenic cleavage assay. To prepare ClickBival(TCO), DMSO stock solutions of P4-DAP-(PA2Tz) (P4-Tz, 15.5 mM) and TCO-Gly2-P12 (TCO-P12, 14.5 mM) were combined at a 1:1 ratio and allowed to react for 3 minutes. LCMS analysis confirmed quantitative conversion to the ligated product (ESI-MS [M-2H]m/z calcd. 1235.51 for C₁₁₄H160N26036, found 1235.41). In parallel, human thrombin (Sigma Aldrich, T6884) and N-p-tosyl-Gly-Pro-Arg-7-amido-4-methylcoumadin hydrochloride (10 μM, Sigma Aldrich, T0273) were combined in phosphate buffered saline (37° C.) and the fluorescence intensity monitored over time (PTI Quantamaster 400, equipped with a 4-sample holder; Ex: 380 nm, Em: 460 nm). After establishing a baseline rate of intensity change vs time, 1 μL of ClickBival(TCO), P4-Tz, TCO-P12, or DMSO respectively were added to the enzyme-substrate solution (2000 μL), in fluorescence cuvettes equipped with magnetic stirring. After adding the ClickBival(TCO), the rate of fluorescence change immediately declined (97% reduction).

Thrombin inhibition by ClickBival(rTCO) is shown in FIG. 6B. The ability of click-assembled bivalirudin (ClickBival) to inhibit human thrombin was tested in a fluorogenic enzyme cleavage assay. To prepare ClickBival(rTCO), DMSO stock solutions of P4-DAP-(PA2Tz) (P4-Tz, 15.5 mM) and rTCO-Gly2-P12 (rTCO-P12, 14.5 mM) were combined at a 1:1 ratio and allowed to react for 5 minutes. In parallel, N-p-tosyl-Gly-Pro-Arg-7-amido-4-methylcoumadin hydrochloride (10 μM, Sigma Aldrich, T0273) was added to fluorescence cuvettes containing 2 mL of phosphate buffered saline (37° C.) and the fluorescence intensity monitored over time (Ex: 380 nm, Em: 460 nm). After establishing a stable baseline, human thrombin (Sigma Aldrich, T6884) was added to initiate the fluorogenic cleavage reaction. Next, 1 μL of ClickBival(rTCO) or DMSO respectively were added to the magnetically-stirred enzyme/substrate reaction mix, leading to an immediate drop in the rate at which the fluorescence signal was rising. Endpoint LCMS analysis of the ClickBival(rTCO) reaction mixture (DMSO) diluted into PBS revealed complete release/cleavage, with no residual ligated product detectable.

### Example 5—ClickBival Self-Destruction Reaction

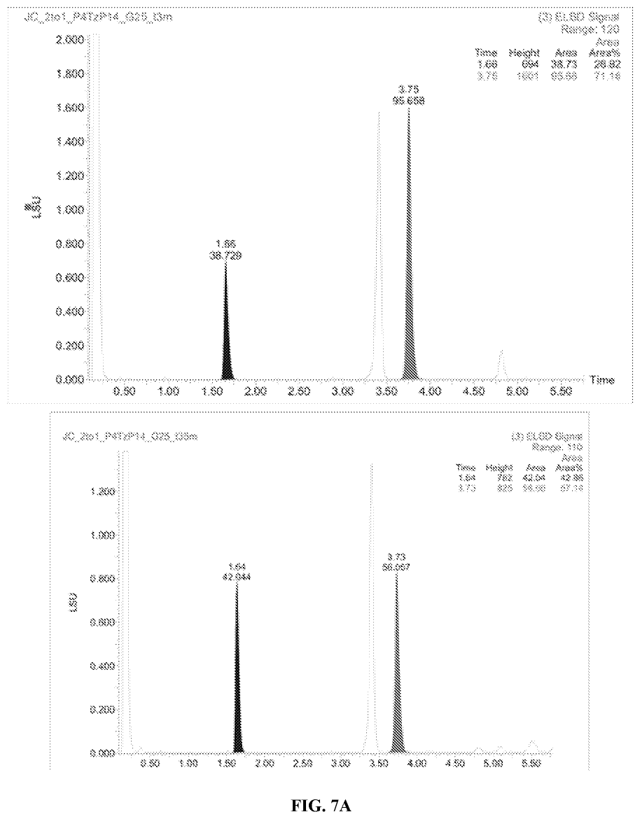

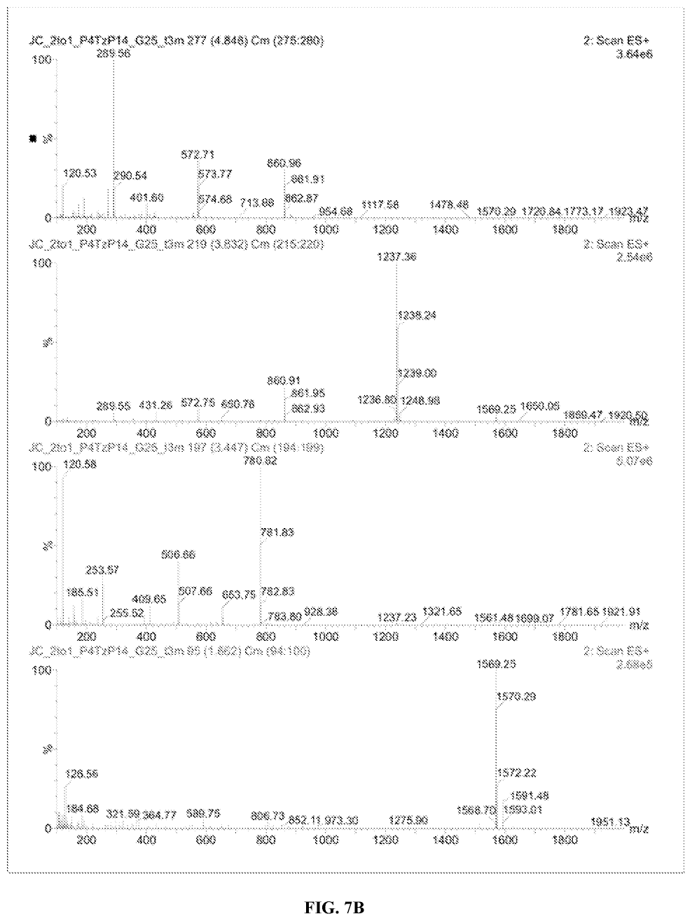

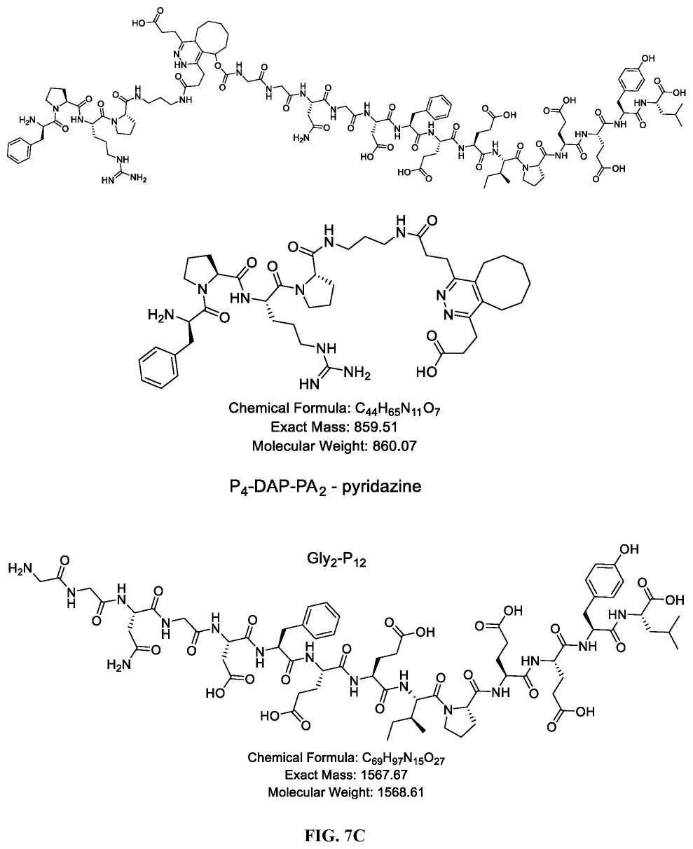

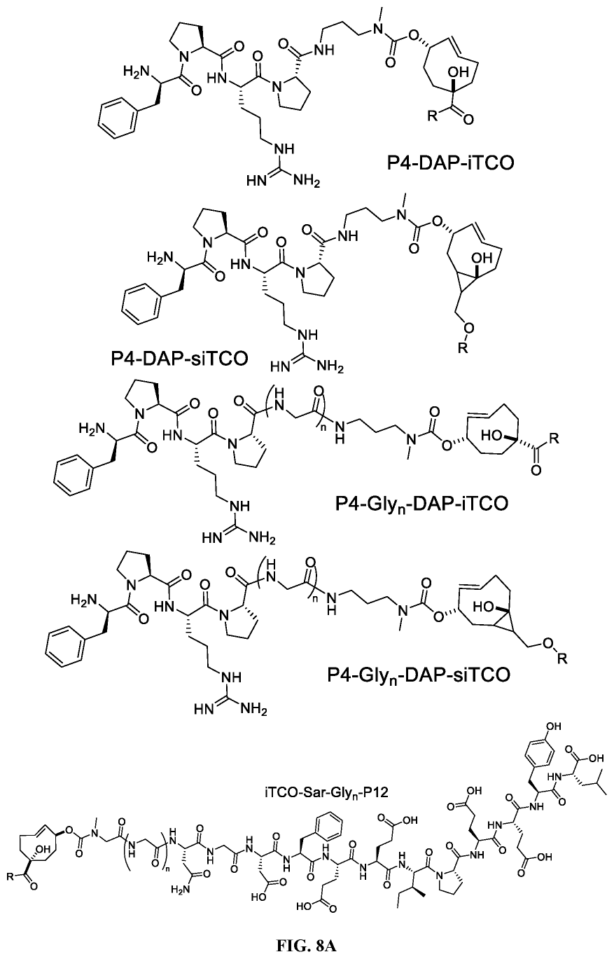

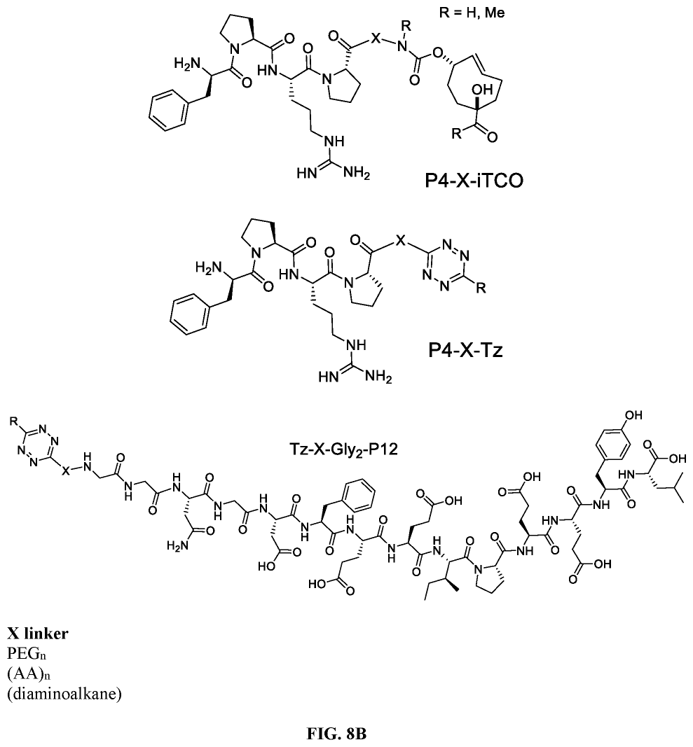

FIG. 7C shows chemical structures of TCO-based click-Bival and corresponding molecular fragments P4-DAP-PA2 and Gly2-P12 resulting from the self-destruction reaction. FIG. 7B contains Mass-spectra showing formation of the reaction fragments. FIG. 7A contains LCMS chromatograms showing self-destruction of TCO-based click-Bival on clinically relevant time-scale.

### Numbered Paragraphs

In some embodiments, the invention of this disclosure can be described by reference to the following numbered paragraphs:

Paragraph 1. A compound of Formula (I):

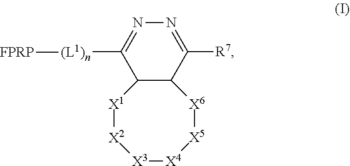

or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and a moiety of formula (i):

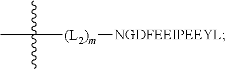

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound of Formula (I) comprises only one moiety of formula (i);
L¹ and L² are each independently selected from N(R^{N}), O, C(O), S, S(O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
m is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

Paragraph 2. The compound of paragraph 1, wherein the compound has formula:

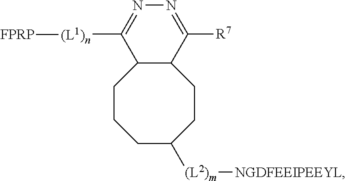

or a pharmaceutically acceptable salt thereof.

Paragraph 3. The compound of paragraph 1, wherein the compound has formula:

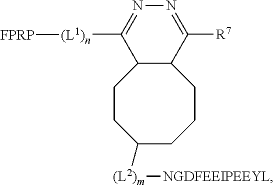

or a pharmaceutically acceptable salt thereof.

Paragraph 4. The compound of paragraph 1, wherein the compound has formula:

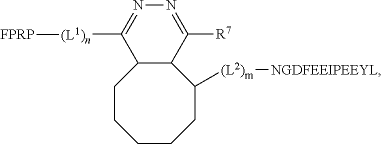

or a pharmaceutically acceptable salt thereof.

Paragraph 5. The compound of paragraph 1, wherein the compound has formula:

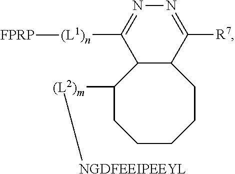

or a pharmaceutically acceptable salt thereof.

Paragraph 6. The compound of paragraph 1, wherein the compound has formula:

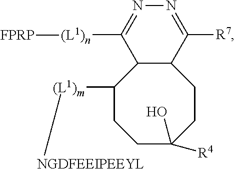

or a pharmaceutically acceptable salt thereof.

Paragraph 7. The compound of paragraph 1, wherein the compound has formula:

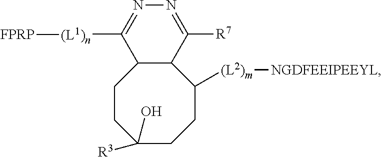

or a pharmaceutically acceptable salt thereof.

Paragraph 8. The compound of paragraph 1, wherein the compound is selected from any one of the following compounds:

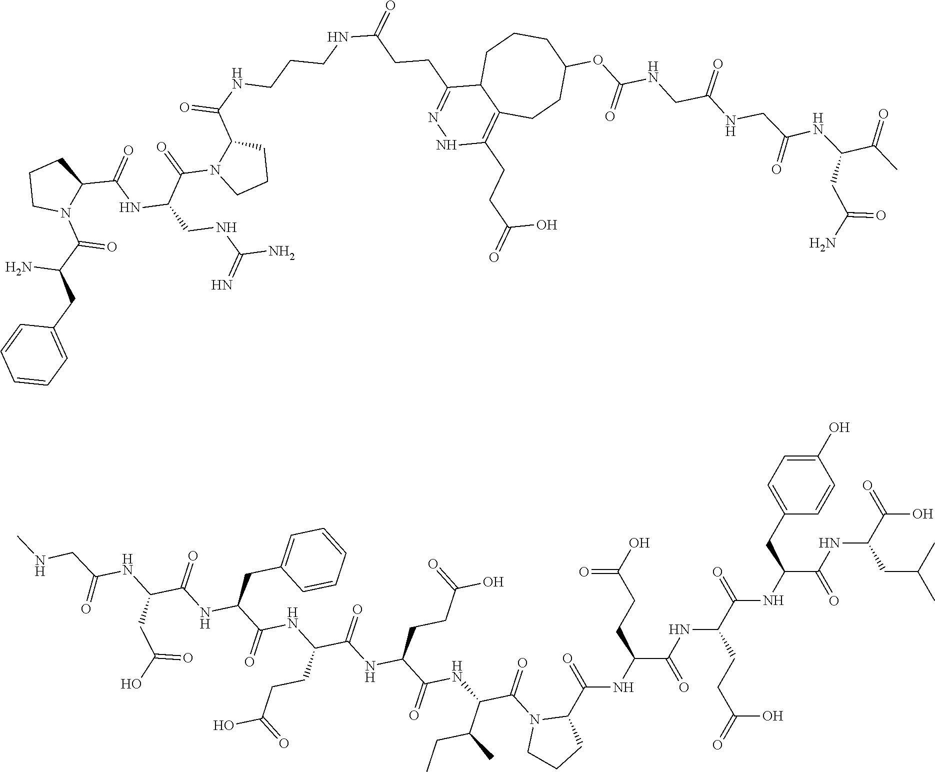

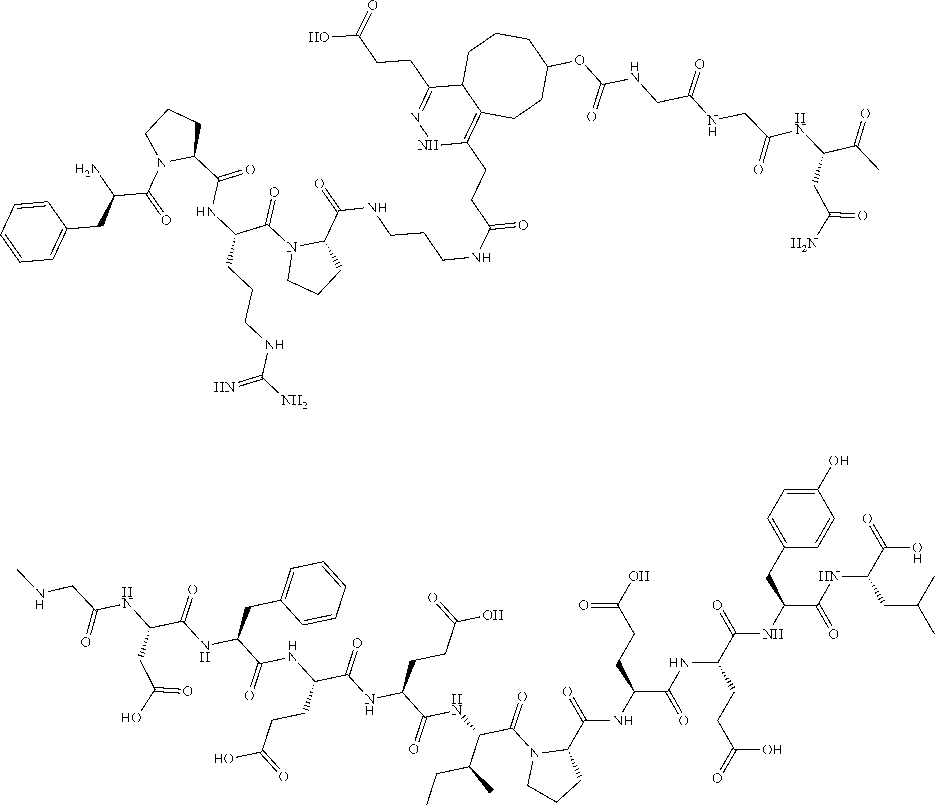

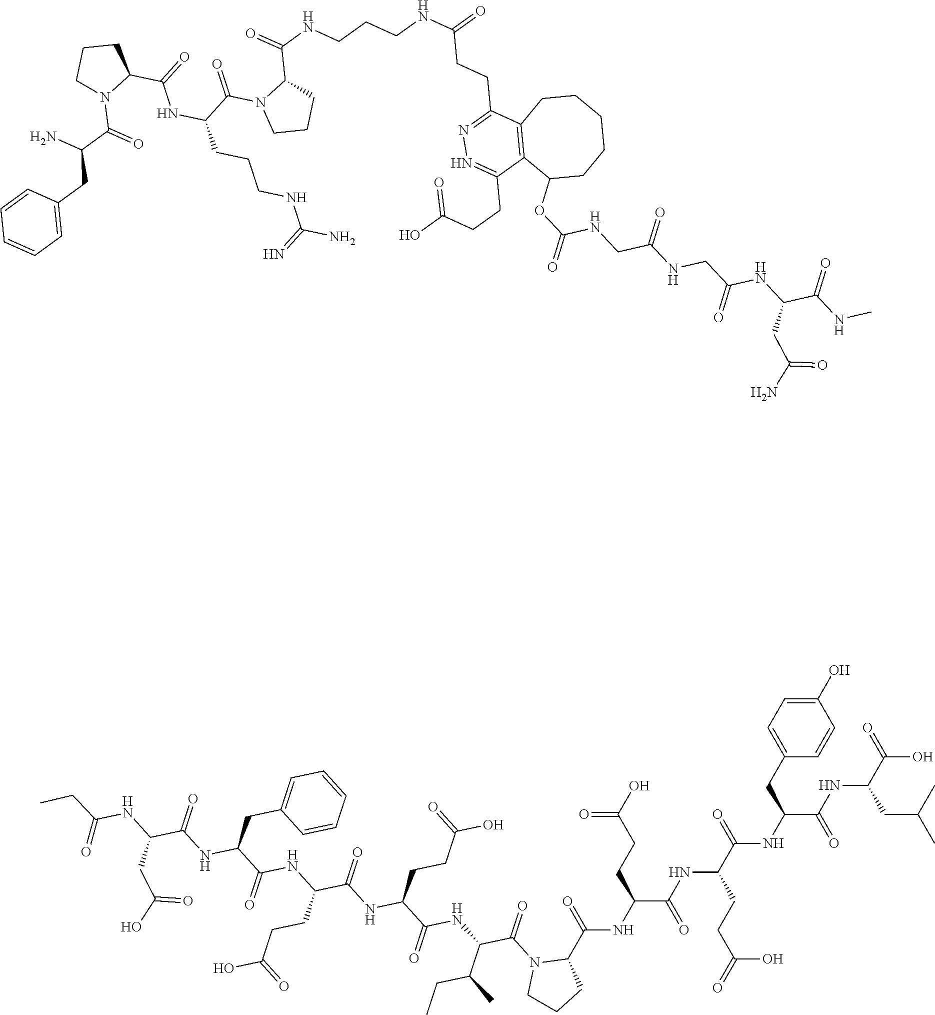

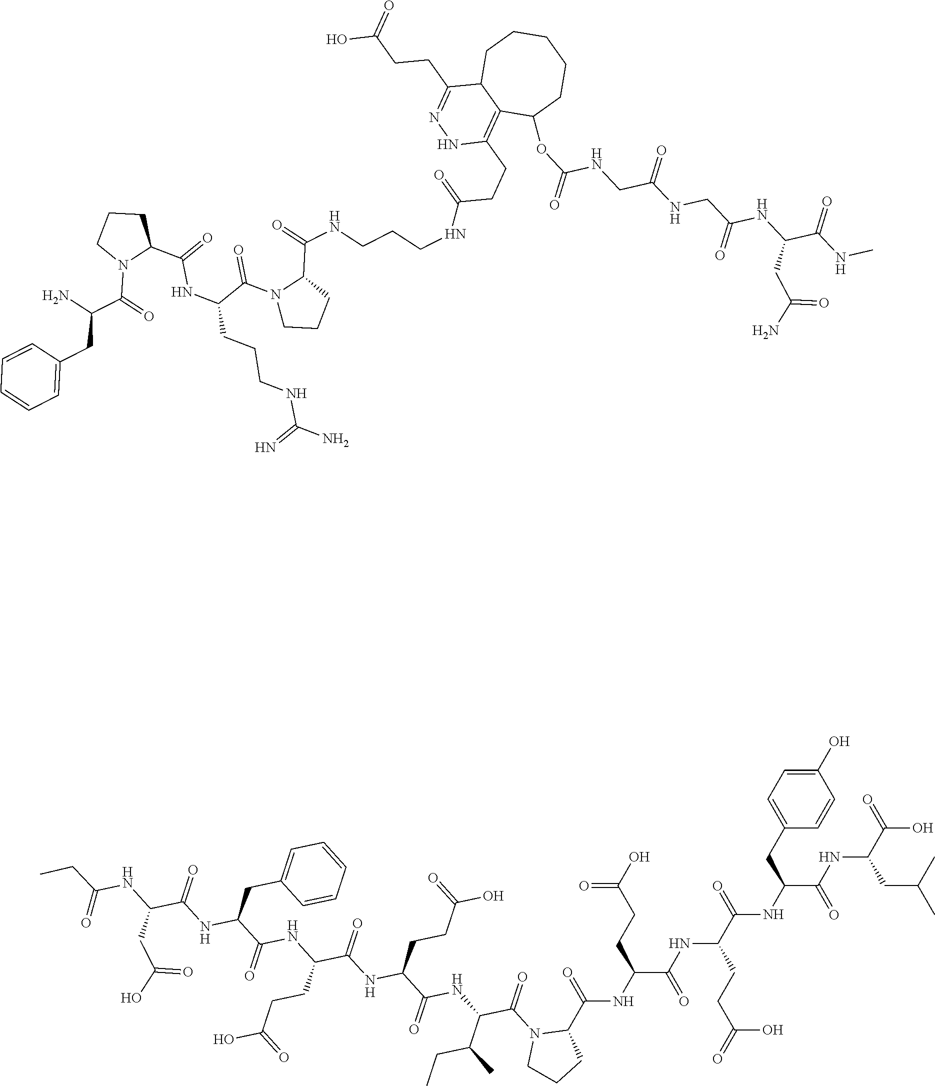

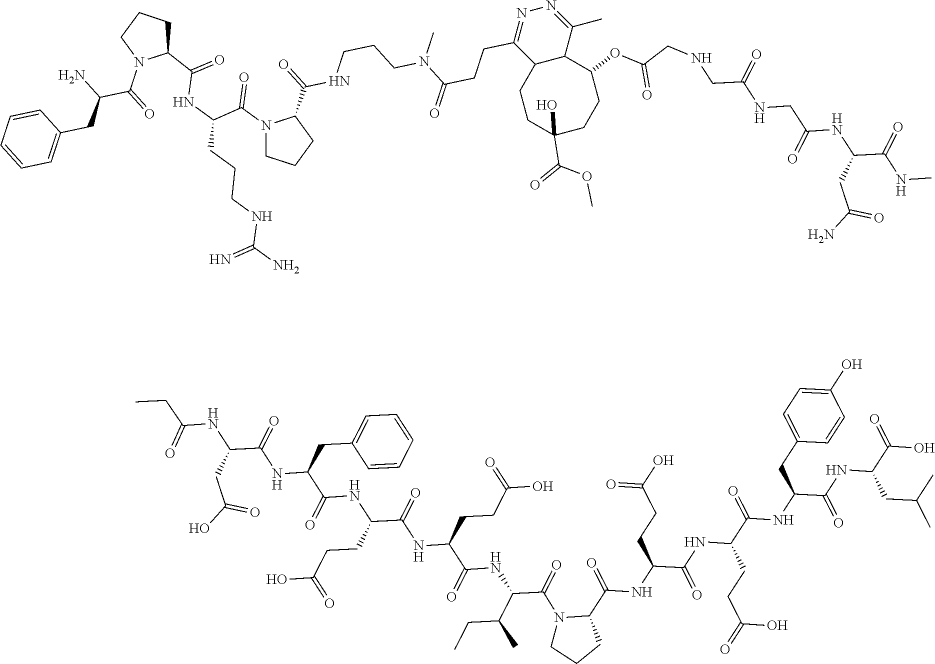

or a pharmaceutically acceptable salt thereof.

Paragraph 9. A compound of Formula (II):

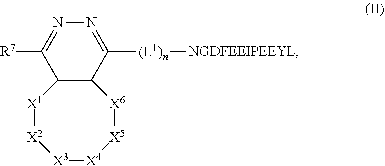

or a pharmaceutically acceptable salt thereof, wherein:
X¹ is selected from 0 and C(R¹)₂;
X² is selected from 0 and C(R²)₂;
X³ is selected from 0 and C(R³)₂;
X⁴ is selected from 0 and C(R⁴)₂;
X⁵ is selected from 0 and C(R⁵)₂;
X⁶ is selected from 0 and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and a moiety of formula (i):

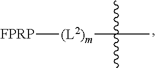

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound of Formula (I) comprises only one moiety of formula (i);
L¹ and L² are each independently selected from N(R^{N}), O, C(O), S, S(O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
m is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

Paragraph 10. The compound of paragraph 9 having formula:

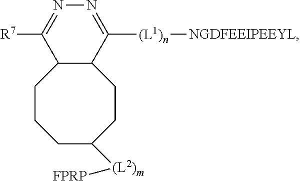

or a pharmaceutically acceptable salt thereof.

Paragraph 11. The compound of paragraph 9 having formula:

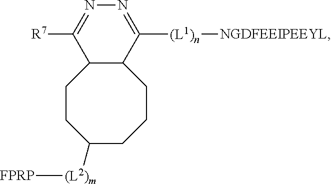

or a pharmaceutically acceptable salt thereof.

Paragraph 12. The compound of paragraph 9 having formula:

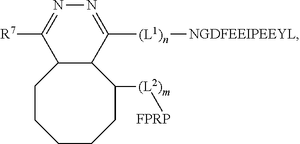

or a pharmaceutically acceptable salt thereof.

Paragraph 13. The compound of paragraph 9 having formula:

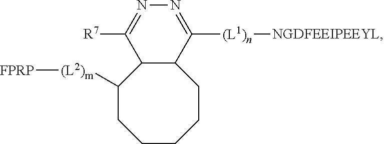

or a pharmaceutically acceptable salt thereof.

Paragraph 14. The compound of paragraph 9 having formula:

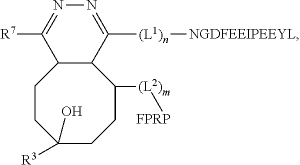

or a pharmaceutically acceptable salt thereof.

Paragraph 15. The compound of paragraph 9 having formula:

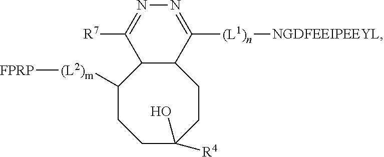

or a pharmaceutically acceptable salt thereof.

Paragraph 16. The compound of paragraph 9, selected from any one of the following compounds:

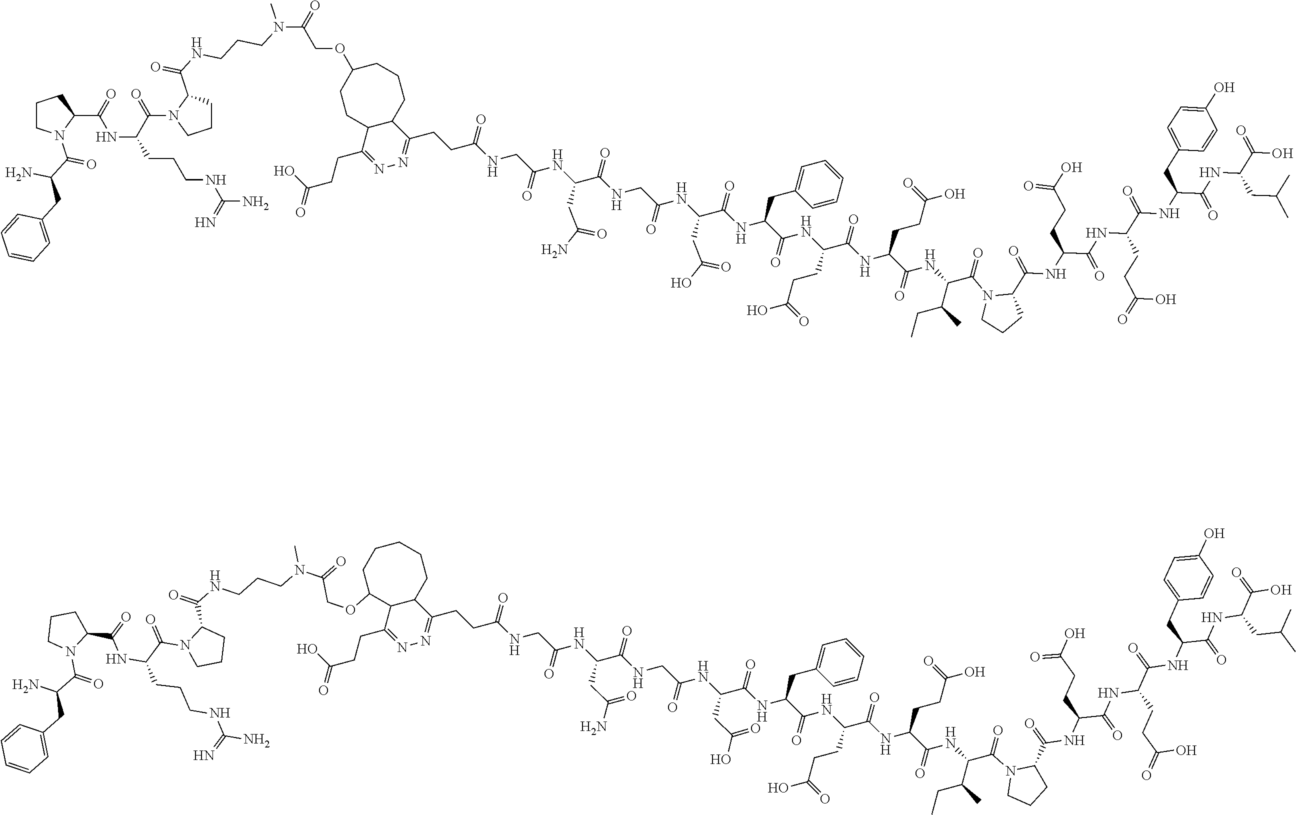

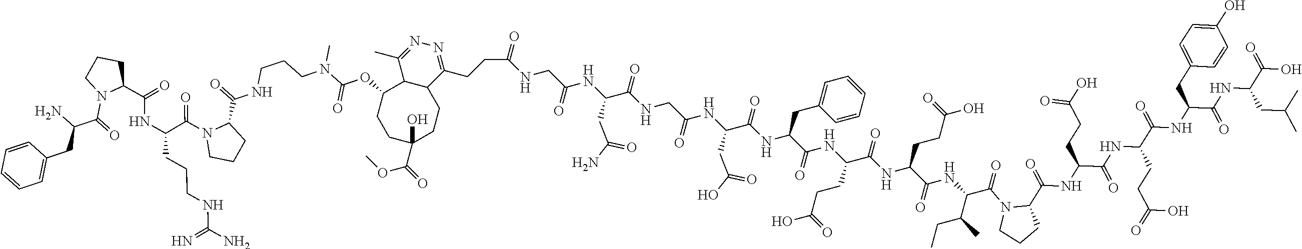

or a pharmaceutically acceptable salt thereof.

Paragraph 17. The compound of any one of paragraphs 1-16, wherein L¹ comprises an amino acid.

Paragraph 18. The compound of paragraph 17, wherein L¹ comprises G₂, G₄, G₆, or G₈.

Paragraph 19. The compound of any one of paragraphs 1-18, wherein L¹ comprises —OC(═O)—, —NHC(═O)—, —C(═O)NH—, or —C(O)O—.

Paragraph 20. The compound ofany one of paragraphs 1-19, wherein L¹ comprises —N(R^{N})—C₁₋₆ alkyleneN(R^{N})—.

Paragraph 21. The compound of any one of paragraphs 1-20, wherein L¹ comprises —(OCH₂CH₂)—, or (CH₂CH₂O)ₓ—, Paragraph 22. The compound of any one of paragraphs 1-21, wherein L² comprises an amino acid.

Paragraph 23. The compound of paragraph 22, wherein L² comprises G₂, G₄, G₆, or G₈.

Paragraph 24. The compound of any one of paragraphs 1-23, wherein L² comprises —OC(═O)—, —NHC(═O)—, —C(═O)N—, or —C(═O)O—.

Paragraph 25. The compound of any one of paragraphs 1-24, wherein L² comprises —N(R^{N})—C₁₋₆ alkylene-N(R^{N})—.

Paragraph 26. The compound of any one of paragraphs 1-25, wherein L² comprises —(OCH₂CH₂)ₓ— or —(CH₂CH₂O)ₓ—.

Paragraph 27. The compound of any one of paragraphs 1-26, wherein R⁷ is C₁₋₆ alkyl, optionally substituted with OH, NH₂, or COOH.

Paragraph 28. The compound of paragraph 27, wherein R⁷ is C₁₋₆ alkyl.

Paragraph 29. The compound of any one of paragraphs 1-26, wherein R⁷ is C₆₋₁₀ aryl or 5-6-membered heteroaryl, each of which is optionally substituted with OH, NH₂, or C(O)OH.

Paragraph 30. A pharmaceutical composition comprising a compound of any one of paragraphs 1-29, or a pharmaceutically acceptable salt thereof, and a pharmaceutically acceptable carrier.

Paragraph 31. A method of treating or preventing coagulation and/or blood clotting in a subject in need thereof, the method comprising administering to the subject a therapeutically effective amount of a compound of any one of paragraphs 1-29, or a pharmaceutically acceptable salt thereof, or a pharmaceutical composition of paragraph 30.

Paragraph 32. The method of paragraph 31, wherein the subject in need thereof has unstable angina undergoing percutaneous transluminal coronary angioplasty.

Paragraph 33. The method of paragraph 32, wherein the subject in need thereof undergoes percutaneous coronary intervention.

Paragraph 34. A compound having Formula (III):

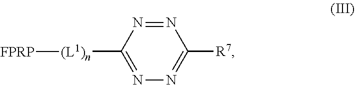

or a pharmaceutically acceptable salt thereof, wherein:
each L¹ is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

Paragraph 35. The compound of paragraph 34, wherein (L¹)ₙ comprises —(OCH₂CH₂)ₓ—, —(CH₂CH₂O)ₓ—, C₁₋₃ alkylene, C(═O), (amino acid)ₙ, and —N(R^{N})—C₁₋₃ alkylene-N(R^{N})—,

Paragraph 36. The compound of paragraph 34, wherein n is selected from 1, 2, 3, 4, or 5.

Paragraph 37. The compound of paragraph 34, wherein R⁷ is C₁₋₆ alkyl, optionally substituted with OH, NH₂, or COOH.

Paragraph 38. The compound of paragraph 37, wherein R⁷ is C₁₋₆ alkyl.

Paragraph 39. The compound of paragraph 34, wherein R⁷ is C₆₋₁₀ aryl or 5-6-membered heteroaryl, each of which is optionally substituted with OH, NH₂, or C(O)OH.

Paragraph 40. The compound of paragraph 34, selected from any one of the following compounds:

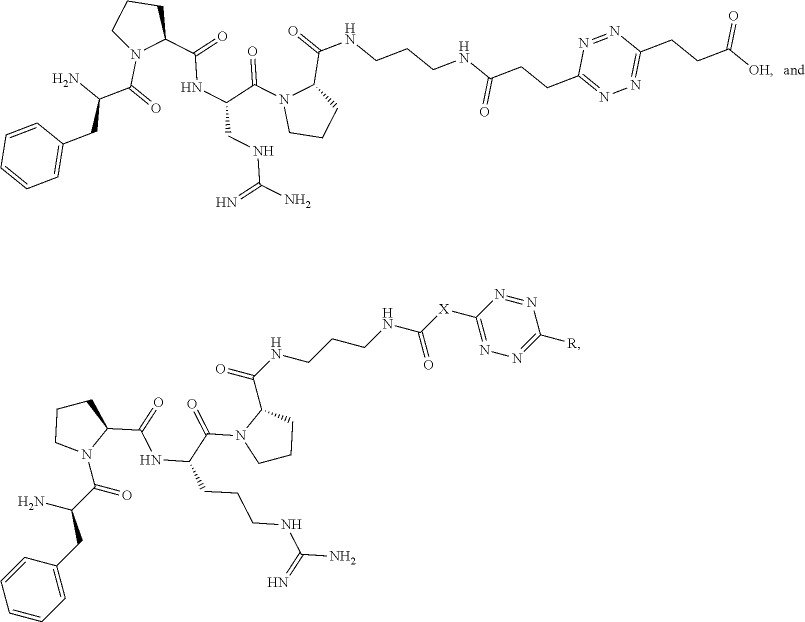

wherein X is selected from methylene, ethylene, and propylene, and R is selected from methyl, 2-carboxyethyl, and 2-aminoethyl,
or a pharmaceutically acceptable salt thereof.

Paragraph 41. A pharmaceutical composition comprising a compound of any one of paragraphs 34-40, or a pharmaceutically acceptable salt thereof, and a pharmaceutically acceptable carrier.

Paragraph 42. A compound of Formula (IV):

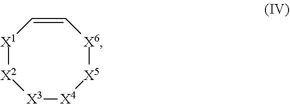

or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and a moiety of formula (i):

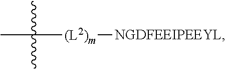

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound of Formula (IV) comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl.

Paragraph 43. The compound of paragraph 42, wherein the compound has formula:

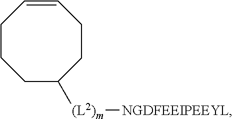

or a pharmaceutically acceptable salt thereof.

Paragraph 44. The compound of paragraph 42, wherein the compound has formula:

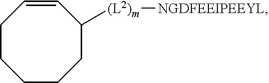

or a pharmaceutically acceptable salt thereof.

Paragraph 45. The compound of paragraph 42, wherein the compound has

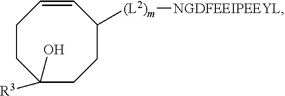

or a pharmaceutically acceptable salt thereof.

Paragraph 46. The compound of any one of paragraphs 42-45, wherein L² comprises —OC(═O)—, —NHC(═O)—, —C(═O)NH—, or —C(═O)O—.

Paragraph 47. The compound of any one of paragraphs 42-46, wherein L² comprises —N(R^{N})—C₁₋₆ alkylene-N(R^{N})—.

Paragraph 48. The compound of any one of paragraphs 42-47, wherein L² comprises —(OCH₂CH₂)ₓ— or —(CH₂CH₂O)ₓ—.

Paragraph 49. The compound of any one of paragraphs 42-48, wherein m is selected from 1, 2, 3, 4, and 5.

Paragraph 50. The compound of paragraph 42, wherein the compound of Formula (IV) is selected from any one of the following compounds:

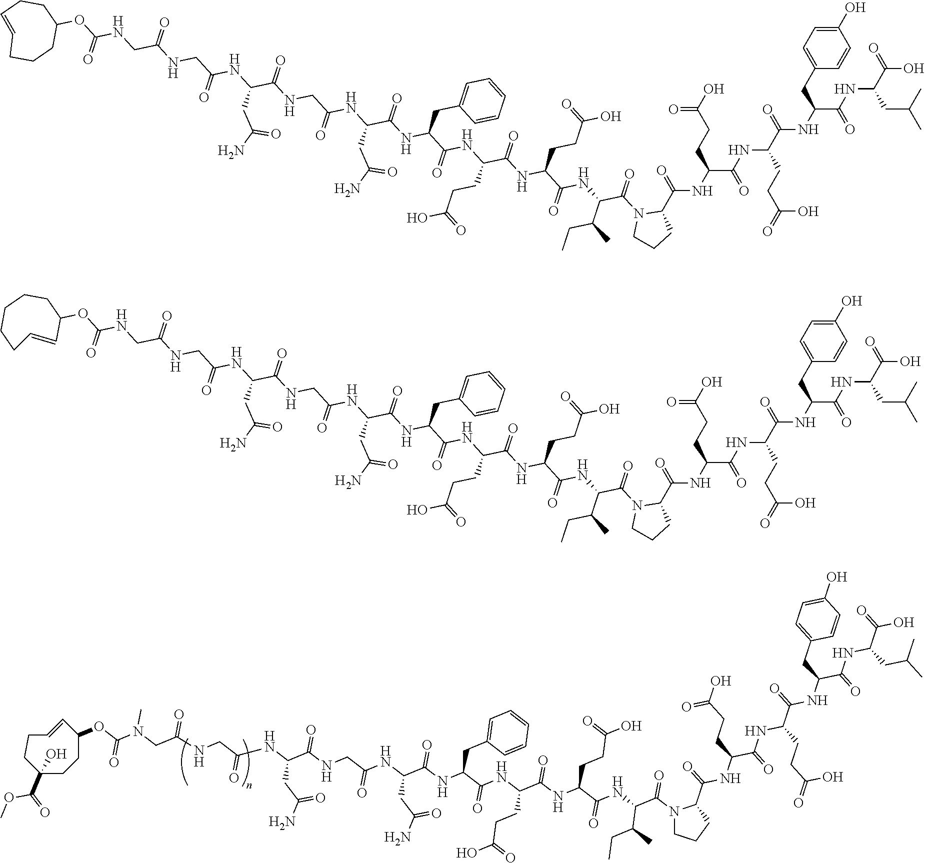

or a pharmaceutically acceptable salt thereof.

Paragraph 51. A pharmaceutical composition comprising a compound of any one of paragraphs 42-50, or a pharmaceutically acceptable salt thereof, and a pharmaceutically acceptable carrier.

Paragraph 52. A method of making a compound of Formula (I) as recited in paragraph 1, the method comprising reacting a compound of Formula (III), or a pharmaceutically acceptable salt thereof, as recited in paragraph 34, with a compound of formula (IV), or a pharmaceutically acceptable salt thereof, as recited in paragraph 42, to obtain the compound of Formula (I), or a pharmaceutically acceptable salt thereof.

Paragraph 53. The method of paragraph 52, wherein the reacting is carried out in a physiologically acceptable medium.

Paragraph 54. The method of paragraph 53, wherein the physiologically acceptable medium comprises a buffer comprising pH from about 5.5 to about 8, a saline, a dextrose solution, or an aqueous solution suitable for injection or infusion.

Paragraph 55. A method of treating or preventing coagulation and/or blood clotting in a subject in need thereof, the method comprising administering to the subject a therapeutically effective amount of a compound of Formula (III), or a pharmaceutically acceptable salt thereof, as recited in any one of paragraphs 34-40, or a pharmaceutical composition of paragraph 41, in combination with a therapeutically effective amount of a compound of Formula (IV), or a pharmaceutically acceptable salt thereof, as recited in any one of paragraphs 42-50, or a pharmaceutical composition of paragraph 51.

Paragraph 56. The method of paragraph 55, wherein the subject in need thereof has unstable angina undergoing percutaneous transluminal coronary angioplasty.

Paragraph 57. The method of paragraph 56, wherein the subject in need thereof undergoes percutaneous coronary intervention.

Paragraph 58. The method of any one of paragraphs 55-57, comprising administering the compound of Formula (III), or a pharmaceutically acceptable salt thereof, and the compound of Formula (IV), or a pharmaceutically acceptable salt thereof, in about stoichiometric amounts.

Paragraph 59. A compound of Formula (V):

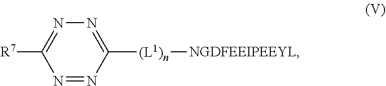

or a pharmaceutically acceptable salt thereof, wherein:
each L¹ is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

Paragraph 60. The compound of paragraph 59, wherein (L¹)ₙ comprises —(OCH₂CH₂)ₓ—, —(CH₂CH₂O)ₓ—, C₁₋₃ alkylene, C(═O), (amino acid)ₙ, and —N(R^{N})—C₁₋₃ alkylene-N(R^{N})—,

Paragraph 61. The compound of paragraph 59, wherein n is selected from 1, 2, 3, 4, or 5.

Paragraph 62. The compound of paragraph 59, wherein R⁷ is C₁₋₆ alkyl, optionally substituted with OH, NH₂, or COOH.

Paragraph 63. The compound of paragraph 62, wherein R⁷ is C₁₋₆ alkyl.

Paragraph 64. The compound of paragraph 59, wherein R⁷ is C₆₋₁₀ aryl or 5-6-membered heteroaryl, each of which is optionally substituted with OH, NH₂, or C(O)OH.

Paragraph 65. The compound of paragraph 59, selected from any one of the following compounds:

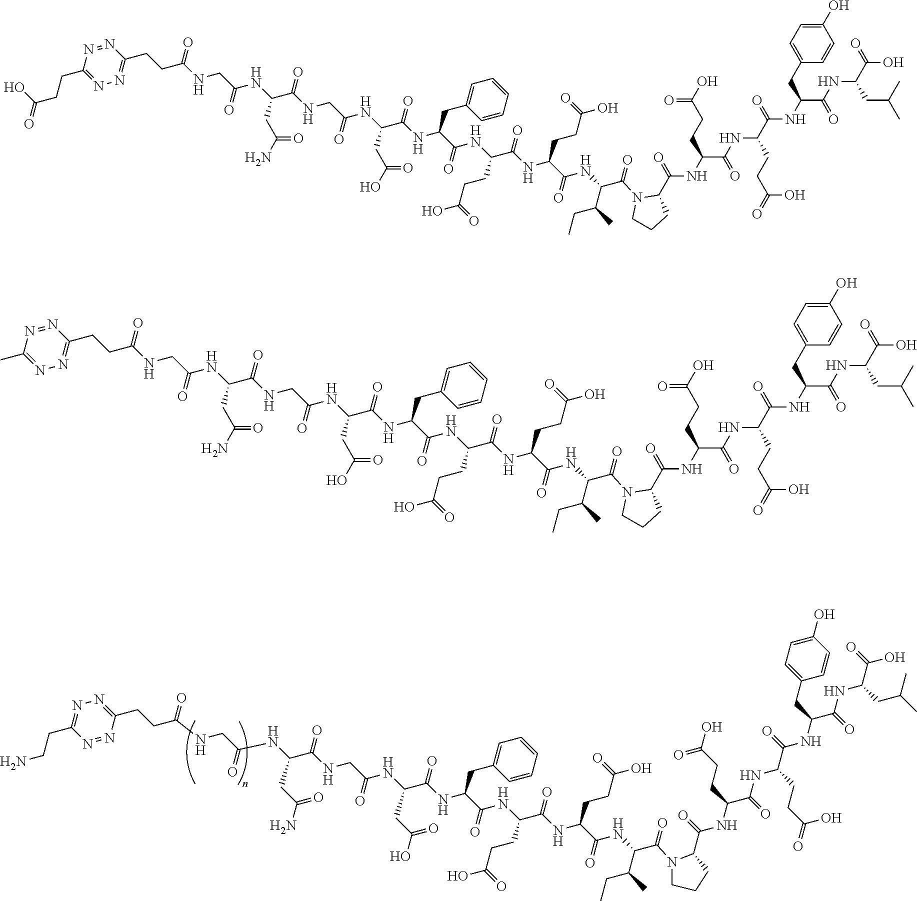

wherein n is 2, 4, or 6,
or a pharmaceutically acceptable salt thereof.

Paragraph 66. A pharmaceutical composition comprising a compound of any one of paragraphs 59-65, or a pharmaceutically acceptable salt thereof, and a pharmaceutically acceptable carrier.

Paragraph 67. A compound of Formula (VI):

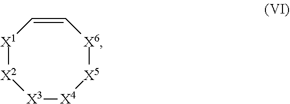

or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and a moiety of formula (i):

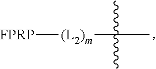

or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound of Formula (VI) comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl.

Paragraph 68. The compound of paragraph 67, wherein the compound of Formula (VI) has formula:

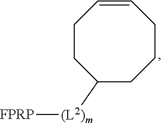

or a pharmaceutically acceptable salt thereof.

Paragraph 69. The compound of paragraph 67, wherein the compound of Formula (VI) has formula:

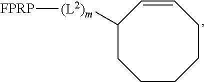

or a pharmaceutically acceptable salt thereof.

Paragraph 70. The compound of paragraph 67, wherein the compound of Formula (VI) has formula:

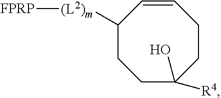

or a pharmaceutically acceptable salt thereof.

Paragraph 71. The compound of any one of paragraphs 67-70, wherein L² comprises —OC(═O)—, —NHC(═O)—, —C(═O)NH—, or —C(═O)O—.

Paragraph 72. The compound of any one of paragraphs 67-71, wherein L² comprises —N(R^{N})—C₁₋₆ alkylene-N(R^{N})—.

Paragraph 73. The compound of any one of paragraphs 67-72, wherein L² comprises —(OCH₂CH₂)ₓ— or —(CH₂CH₂O)ₓ—.

Paragraph 74. The compound of any one of paragraphs 67-73, wherein m is selected from 1, 2, 3, 4, and 5.

Paragraph 75. The compound of paragraph 67, selected from any one of the following compounds:

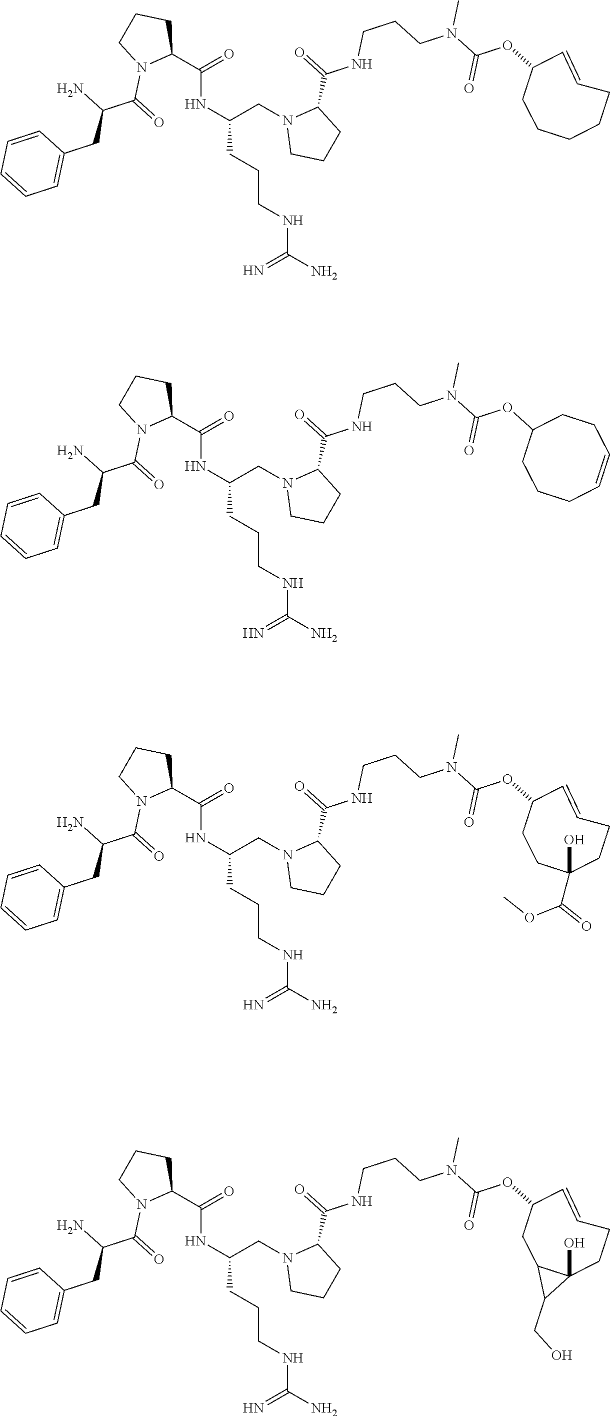

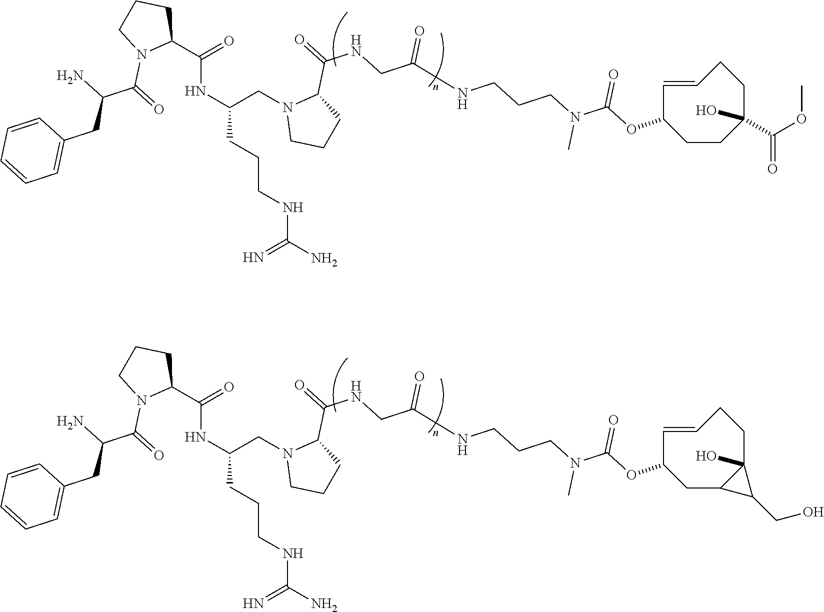

wherein n is 2, 4, or 6,
or a pharmaceutically acceptable salt thereof.

Paragraph 76. A pharmaceutical composition comprising a compound of any one of paragraphs 67-75, or a pharmaceutically acceptable salt thereof, and a pharmaceutically acceptable carrier.

Paragraph 77. A method of making a compound of Formula (II), or a pharmaceutically acceptable salt thereof, as recited in paragraph 9, the method comprising reacting a compound of Formula (V), or a pharmaceutically acceptable salt thereof, as recited in paragraph 59, with a compound of formula (VI), or a pharmaceutically acceptable salt thereof, as recited in paragraph 67, to obtain the compound of Formula (II), or a pharmaceutically acceptable salt thereof.

Paragraph 78. The method of paragraph 77, wherein the reacting is carried out in a physiologically acceptable medium.

Paragraph 79. The method of paragraph 78, wherein the physiologically acceptable medium comprises a buffer comprising pH from about 5.5 to about 8, a saline, a dextrose solution, or an aqueous solution suitable for injection or infusion.

Paragraph 80. A method of treating or preventing coagulation and/or blood clotting in a subject in need thereof, the method comprising administering to the subject a therapeutically effective amount of a compound of Formula (V), or a pharmaceutically acceptable salt thereof, as recited in any one of paragraphs 59-65, or a pharmaceutical composition of paragraph 66 in combination with a therapeutically effective amount of a compound of Formula (VI), or a pharmaceutically acceptable salt thereof, as recited in any one of paragraphs 67-75, or a pharmaceutical composition of paragraph 76.

Paragraph 81. The method of paragraph 55, wherein the subject in need thereof has unstable angina undergoing percutaneous transluminal coronary angioplasty.

Paragraph 82. The method of paragraph 56, wherein the subject in need thereof undergoes percutaneous coronary intervention.

Paragraph 83. The method of any one of paragraphs 80-82, comprising administering the compound of Formula (V), or a pharmaceutically acceptable salt thereof, and the compound of Formula (VI), or a pharmaceutically acceptable salt thereof, in about stoichiometric amounts.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound selected from: 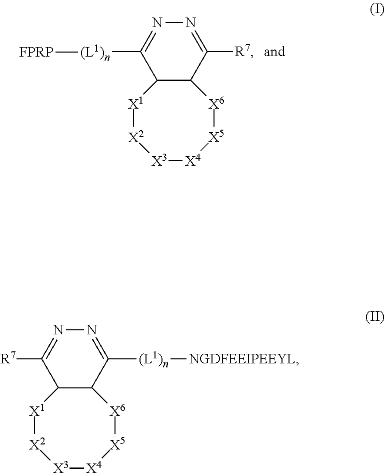
or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and
a moiety of formula (i): 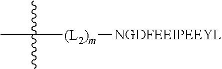
if the compound is a compound of Formula (I), or
a moiety of formula (i): 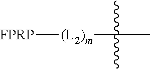
if the compound is a compound of Formula (II),
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound comprises only one moiety of formula (i);
L¹ and L² are each independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
m is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

2. The compound of claim 1, wherein the compound of Formula (I) is selected from any one of the following formulae: 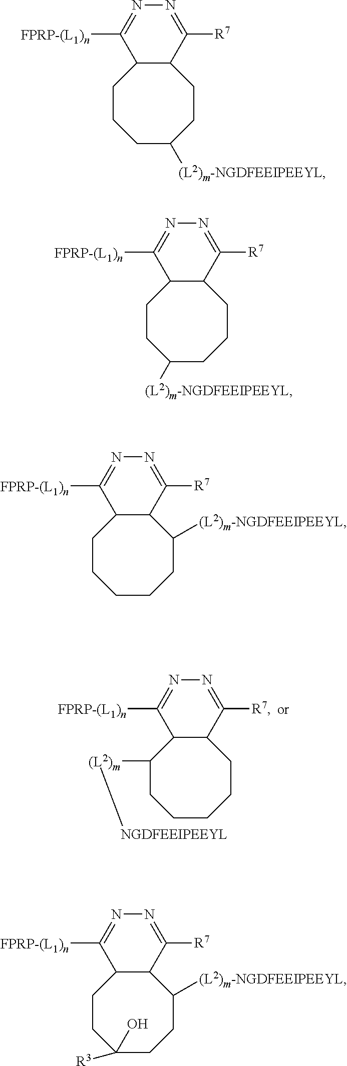
or a pharmaceutically acceptable salt thereof.

3. The compound of claim 1, wherein the compound of Formula (I) is selected from: 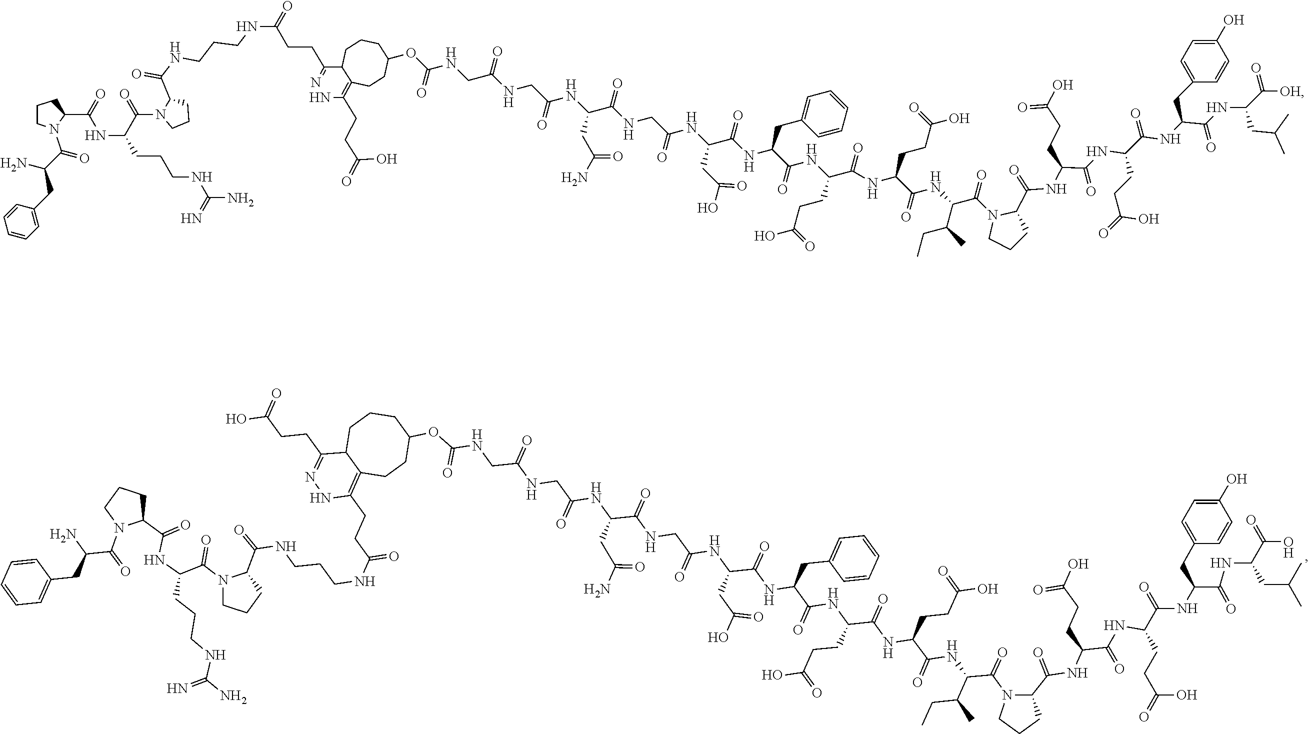 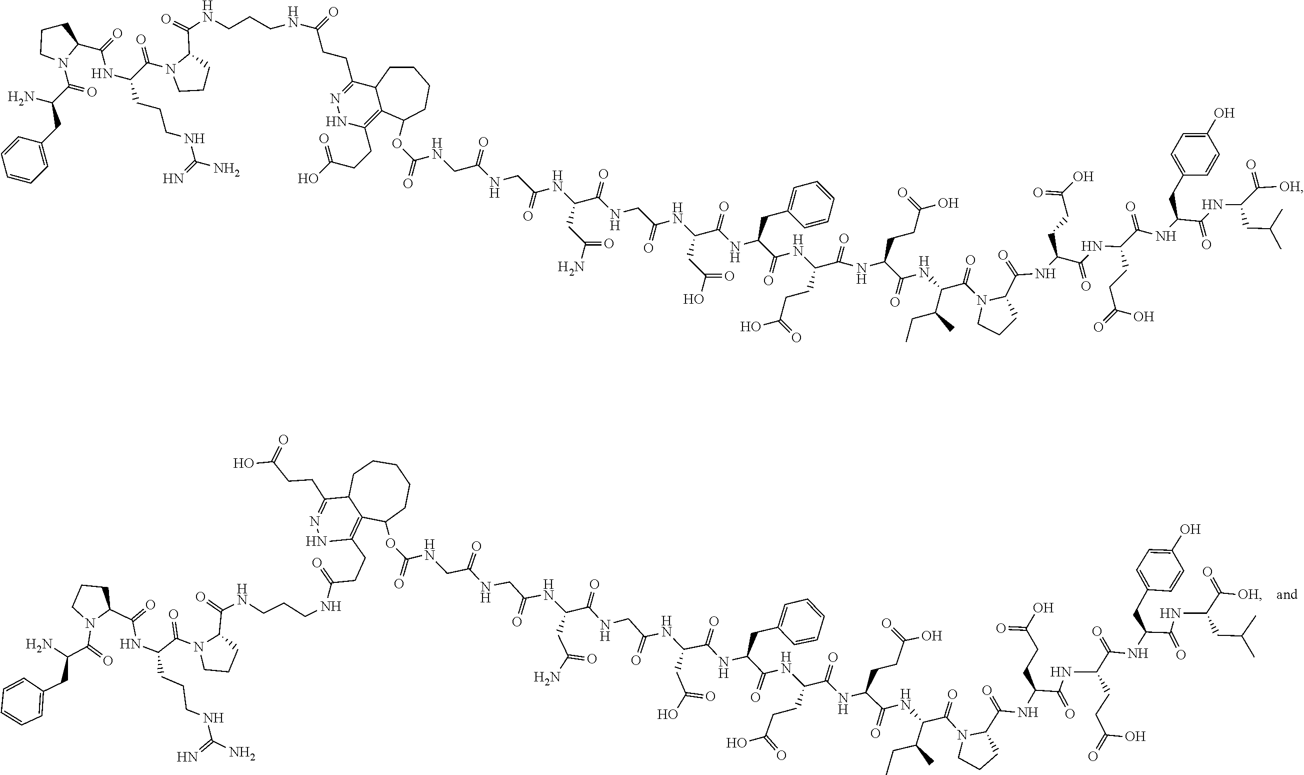 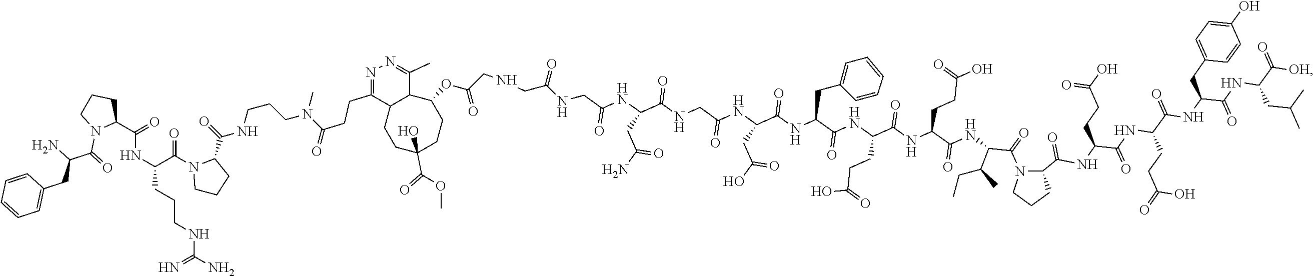
or a pharmaceutically acceptable salt thereof.

4. The compound of claim 1, wherein the compound of Formula (II) is selected from any one of the following formulae: 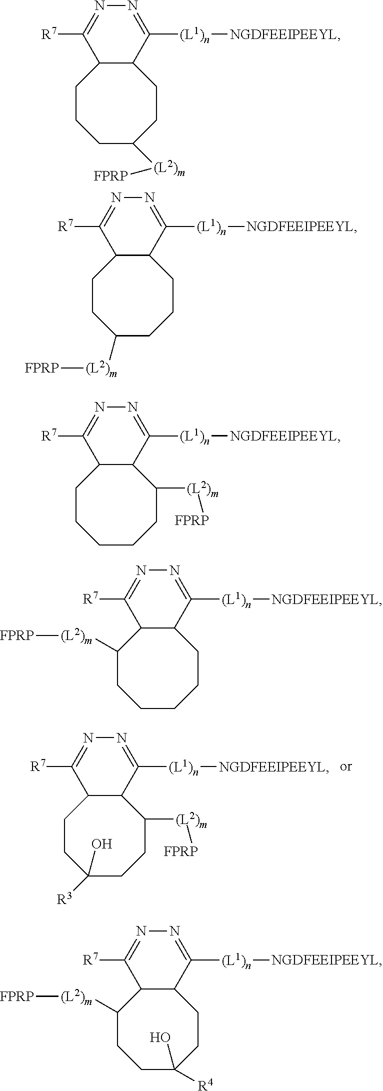
or a pharmaceutically acceptable salt thereof.

5. The compound of claim 1, wherein the compound of Formula (II) is selected from: 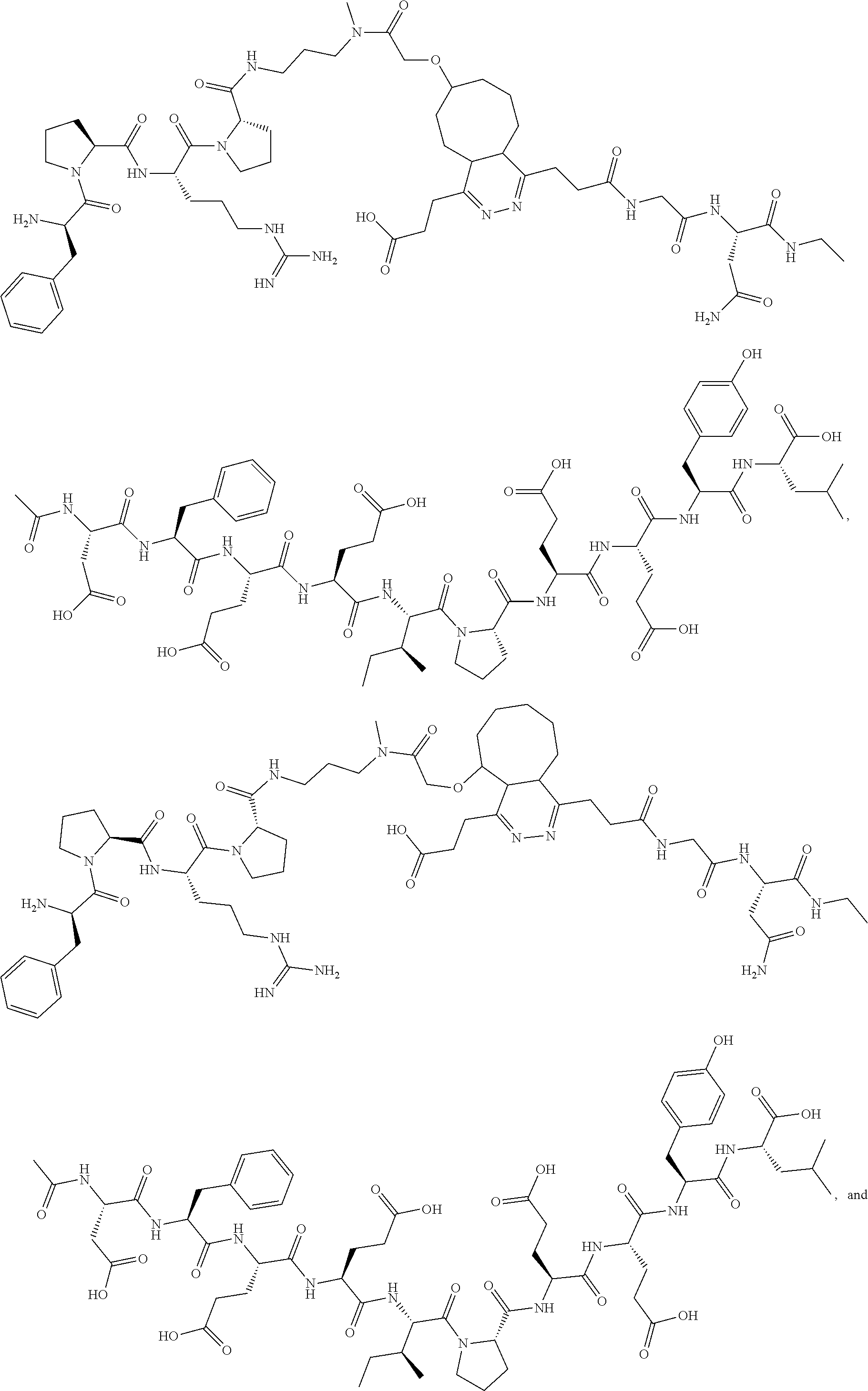 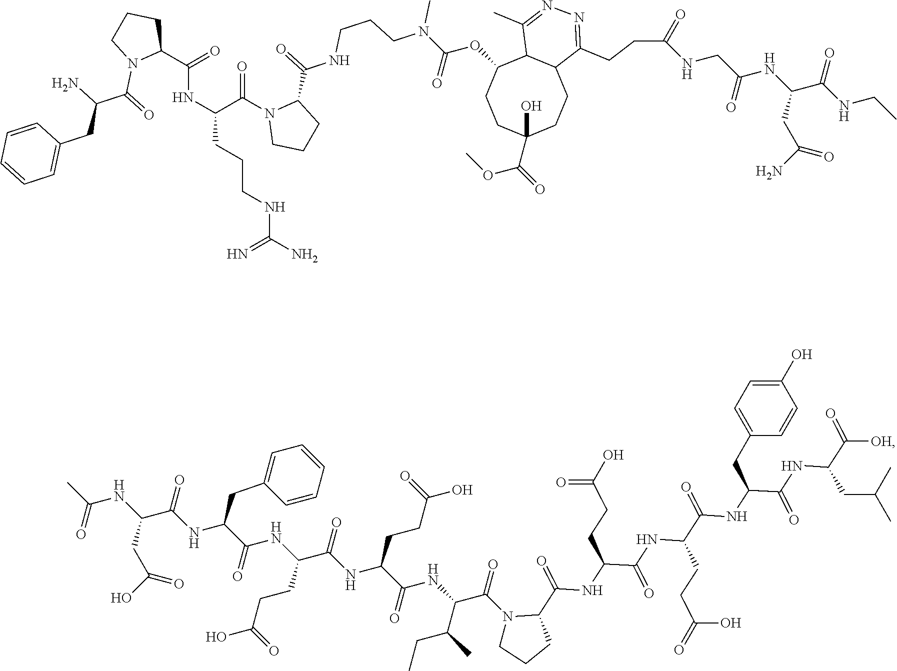
or a pharmaceutically acceptable salt thereof.

6. A pharmaceutical composition comprising a compound of claim 1, or a pharmaceutically acceptable salt thereof, and a pharmaceutically acceptable carrier.

7. A method of treating or preventing coagulation and/or blood clotting in a subject in need thereof, the method comprising administering to the subject a therapeutically effective amount of a compound of claim 1, or a pharmaceutically acceptable salt thereof.

8. The method of claim 7, wherein the subject in need thereof has unstable angina undergoing percutaneous transluminal coronary angioplasty and/or undergoes percutaneous coronary intervention.

9. A compound selected from: 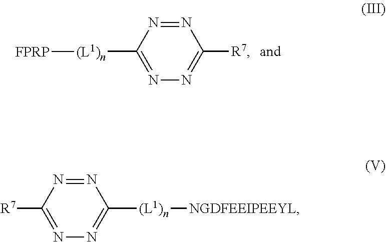
or a pharmaceutically acceptable salt thereof, wherein:
each L¹ is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂₋₄ alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy.

10. The compound of claim 9, wherein the compound of Formula (III) is selected from: 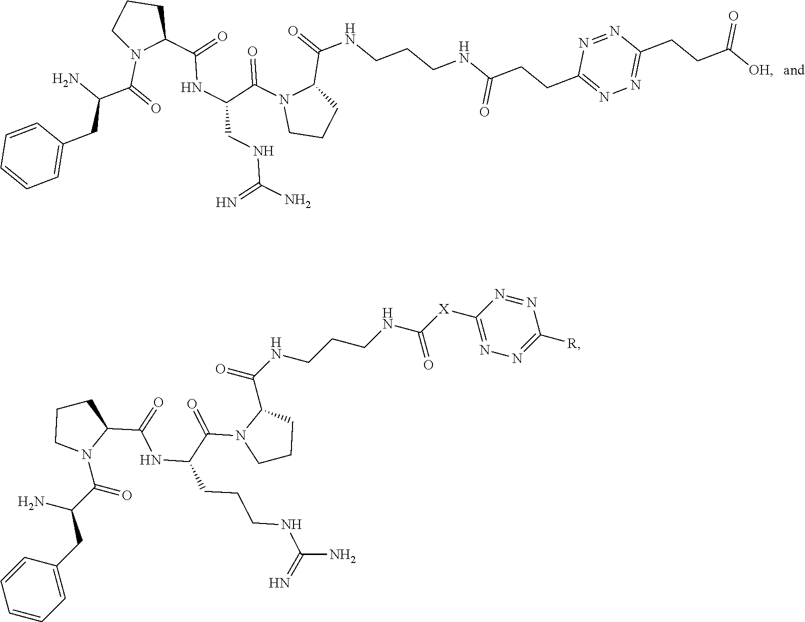
wherein X is selected from methylene, ethylene, and propylene, and R is selected from methyl, 2-carboxyethyl, and 2-aminoethyl,
or a pharmaceutically acceptable salt thereof.

11. The compound of claim 9, wherein the compound of Formula (V) is selected from: 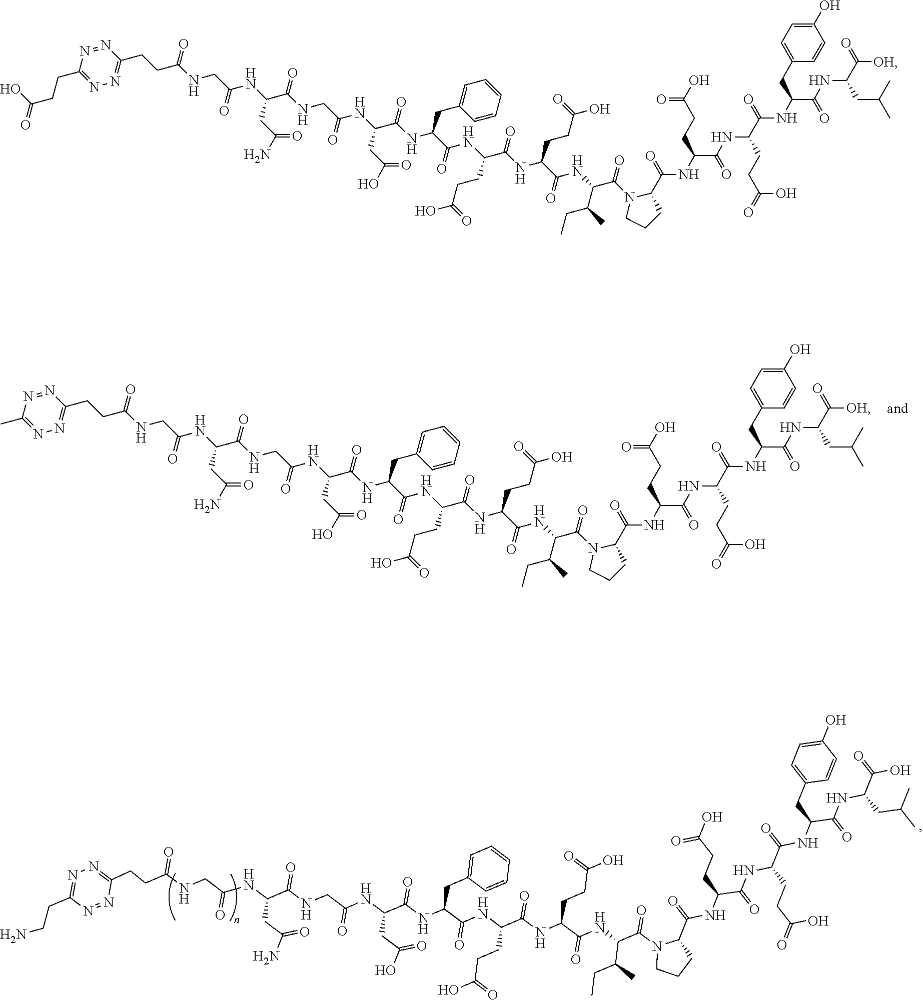
wherein n is 2, 4, or 6, or a pharmaceutically acceptable salt thereof.

12. A pharmaceutical composition comprising a compound of of claim 9, or a pharmaceutically acceptable salt thereof, and a pharmaceutically acceptable carrier.

13. A compound selected from: 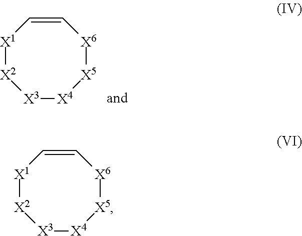
or a pharmaceutically acceptable salt thereof, wherein:
X¹ is selected from 0 and C(RH)₂;
X² is selected from 0 and C(R²)₂;
X³ is selected from 0 and C(R³)₂;
X⁴ is selected from 0 and C(R⁴)₂;
X⁵ is selected from 0 and C(R⁵)₂;
X⁶ is selected from 0 and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and
a moiety of formula (i): 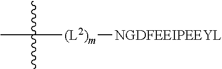
when the compound is the compound of Formula (IV), or
a moiety of formula (i): 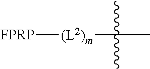
when the compound is the compound of Formula (VI),
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, N12-C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, N12-C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, N12-C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, N12-C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, N12-C₁₋₃ alkylene, and a moiety formula (i);
provided that the compound comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl.

14. The compound of claim 13, wherein the compound of Formula (IV) is selected from any one of the following formulae: 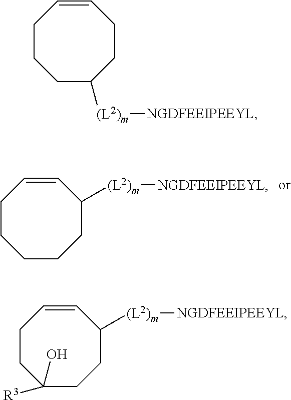
or a pharmaceutically acceptable salt thereof.

15. The compound of claim 13, wherein the compound of Formula (IV) is selected from: 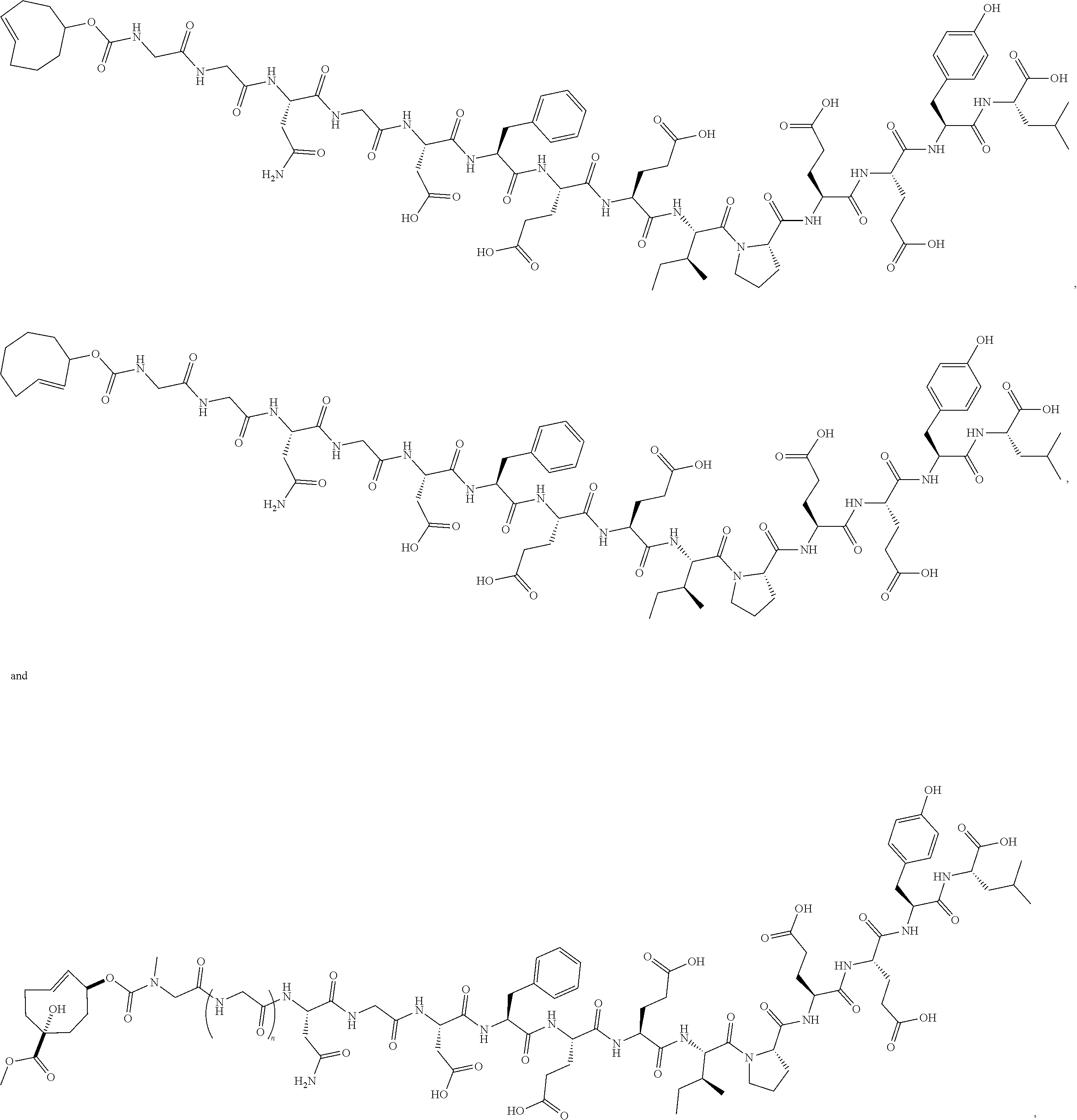
or a pharmaceutically acceptable salt thereof.

16. The compound of claim 13, wherein the compound of Formula (VI) is selected from any one of the following formulae: 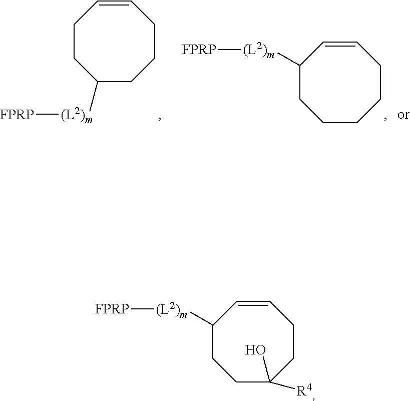
or a pharmaceutically acceptable salt thereof.

17. The compound of claim 13, wherein the compound of Formula (VI) is selected from: 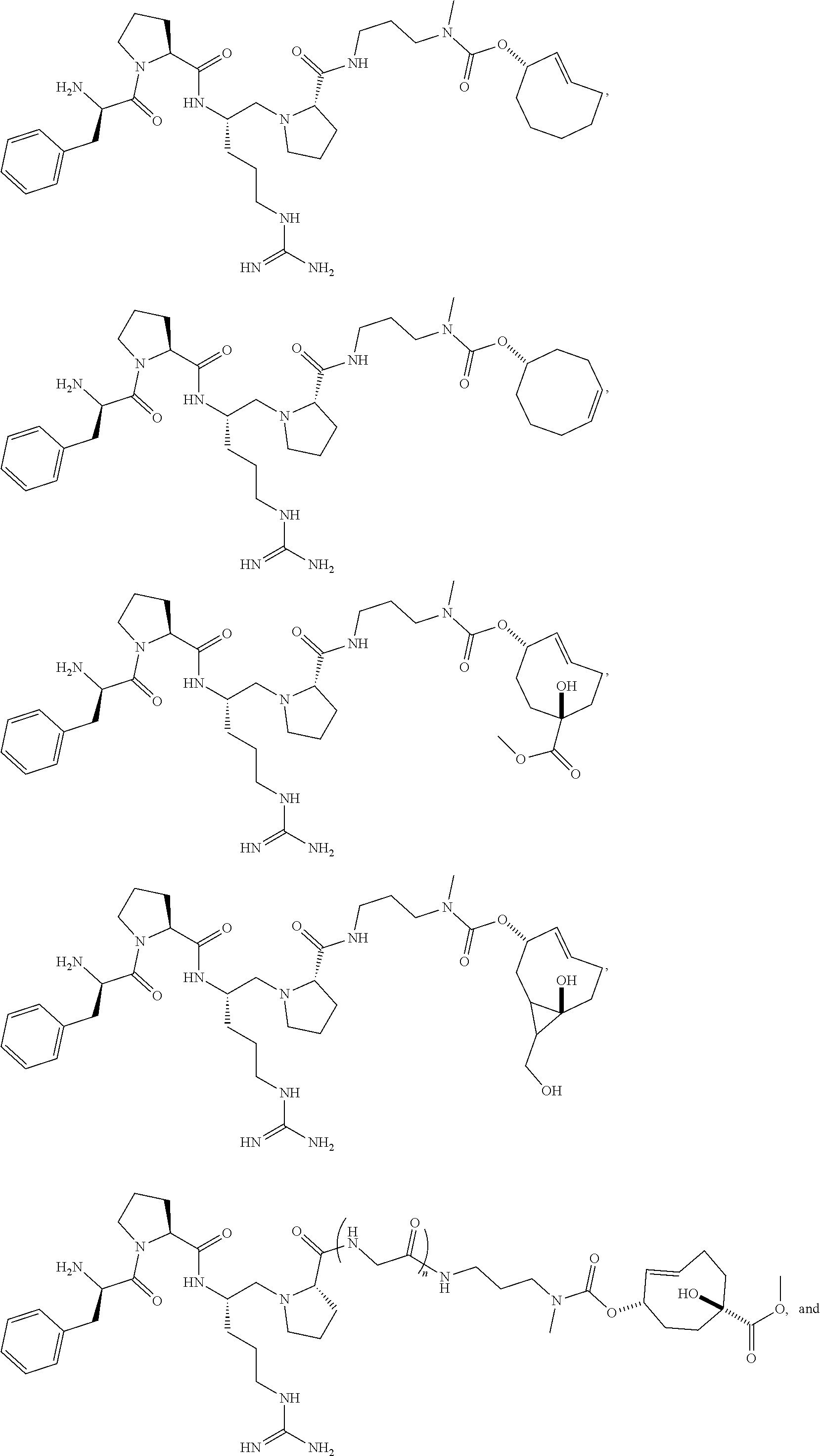 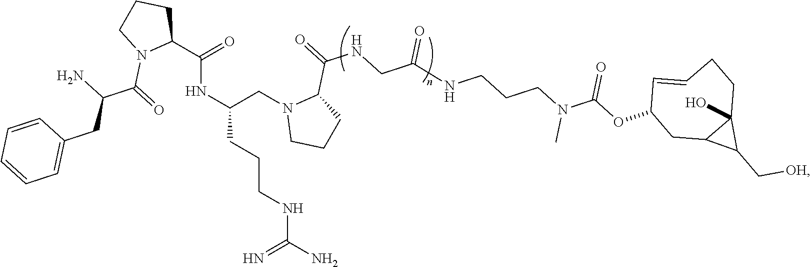
wherein n is 2, 4, or 6, or a pharmaceutically acceptable salt thereof.

18. A pharmaceutical composition comprising a compound of claim 13, or a pharmaceutically acceptable salt thereof, and a pharmaceutically acceptable carrier.

19. A method of making a compound of Formula (I) as recited in claim 1, the method comprising reacting a compound of Formula (III); 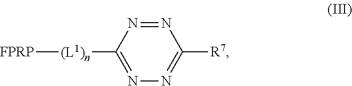
or a pharmaceutically acceptable salt thereof, wherein:
each L¹ is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂-4 alkenyl, C₆-aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy;
with a compound of formula (IV); 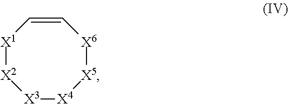
or a pharmaceutically acceptable salt thereof, wherein:
X¹ is selected from 0 and C(R¹)₂;
X² is selected from 0 and C(R²)₂;
X³ is selected from 0 and C(R³)₂;
X⁴ is selected from 0 and C(R⁴)₂;
X⁵ is selected from 0 and C(R⁵)₂;
X⁶ is selected from 0 and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and
a moiety of formula (i): 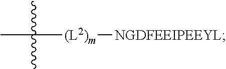
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl; to obtain the compound of Formula (I), or a pharmaceutically acceptable salt thereof.

20. A method of making a compound of Formula (II) as recited in claim 1, the method comprising reacting a compound of Formula (V): 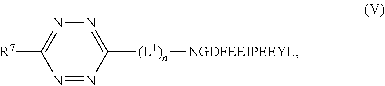
or a pharmaceutically acceptable salt thereof, wherein:
each L¹ is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
n is an integer from 0 to 20;
each x is independently an integer from 1 to 2,000;
each R^{N} is independently selected from H, C₁₋₃ alkyl, and C₁₋₃ haloalkyl, and
R⁷ is selected from H, C₁₋₆ alkyl, C₂₋₄ alkenyl, C₆₋₁₀ aryl, 5-6 membered heterocycloalkyl, and 5-6-membered heteroaryl, wherein said C₁₋₆ alkyl, C₂-4 alkenyl, C₆₋₁₀ aryl, 5-6 membered heterocycloalkyl, and 5-6-membered heteroaryl are each optionally substituted with 1 or 2 substituents independently selected from OH, NH₂, C(O)OH, halo, C₁₋₆ alkoxy, and C₁₋₆ haloalkoxy;
with a compound of formula (VI): 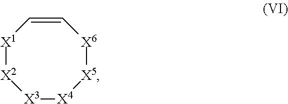
or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and
a moiety of formula (i): 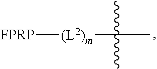
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl;
to obtain the compound of Formula (II), or a pharmaceutically acceptable salt thereof.

21. The method of claim 19, wherein the reacting is carried out in a physiologically acceptable medium.

22. The method of claim 21, wherein the physiologically acceptable medium comprises a buffer comprising pH from about 5.5 to about 8, a saline, a dextrose solution, or an aqueous solution suitable for injection or infusion.

23. A method of treating or preventing coagulation and/or blood clotting in a subject in need thereof, the method comprising administering to the subject a therapeutically effective amount of a compound of Formula (III), or a pharmaceutically acceptable salt thereof, as recited in claim 9, in combination with a therapeutically effective amount of a compound of Formula (IV): 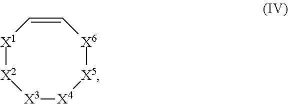
or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and
a moiety of formula (i): 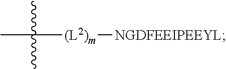
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃10 cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl.

24. The method of claim 23, wherein the subject in need thereof has unstable angina undergoing percutaneous transluminal coronary angioplasty and/or undergoes percutaneous coronary intervention.

25. The method of claim 23, comprising administering the compound of Formula (III), or a pharmaceutically acceptable salt thereof, and the compound of Formula (IV), or a pharmaceutically acceptable salt thereof, in about stoichiometric amounts.

26. A method of treating or preventing coagulation and/or blood clotting in a subject in need thereof, the method comprising administering to the subject a therapeutically effective amount of a compound of Formula (V), or a pharmaceutically acceptable salt thereof, as recited in claim 9, in combination with a therapeutically effective amount of a compound of Formula (VI): 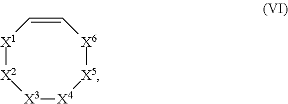
or a pharmaceutically acceptable salt thereof, wherein:
X¹ is selected from O and C(R¹)₂;
X² is selected from O and C(R²)₂;
X³ is selected from O and C(R³)₂;
X⁴ is selected from O and C(R⁴)₂;
X⁵ is selected from O and C(R⁵)₂;
X⁶ is selected from O and C(R⁶)₂;
R¹, R², R³, R⁴, R⁵, and R⁶ are each independently selected from H, OH, NH₂, C(O)OH, C(O)C₁₋₃ alkoxy, C(O)C₁₋₃ alkyl, halo, C₁₋₆ alkyl, C₁₋₆ haloalkyl, C₁₋₃ alkoxy, C₁₋₃ haloalkoxy, and
a moiety of formula (i): 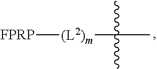
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety of formula (i);
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
or R⁵ and R⁶, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(O)OH, C(O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and a moiety formula (i);
provided that the compound comprises only one moiety of formula (i);
each L² is independently selected from N(R^{N}), O, C(═O), S, S(═O), S(═O)₂, C₁₋₆ alkylene, C₃₋₇ cycloalkylene, C₆₋₁₀ arylene, C₂₋₄ alkenylene, 5-6 membered heterocycloalkylene, 5-6-membered heteroarylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
m is an integer from 0 to 20;
each x is independently an integer from 1 to 2,000; and
each R^{N} is independently selected from H, C₁₋₃ alkyl, and C₁₋₃ haloalkyl.

27. The method of claim 26, wherein the subject in need thereof has unstable angina undergoing percutaneous transluminal coronary angioplasty and/or undergoes percutaneous coronary intervention.

28. The method of claim 26, comprising administering the compound of Formula (V), or a pharmaceutically acceptable salt thereof, and the compound of Formula (VI), or a pharmaceutically acceptable salt thereof, in about stoichiometric amounts.
